(19) United States
(12) Patent Application Publication
(10) Pub. No.: US 2022/0073605 A1
(43) Pub. Date: Mar. 10, 2022

(54) CLAZAKIZUMAB IN THE TREATMENT OF CHRONIC ANTIBODY-MEDIATED REJECTION OF ORGAN TRANSPLANT

(71) Applicant: CEDARS-SINAI MEDICAL CENTER, Los Angeles, CA (US)

(72) Inventors: Stanley C. JORDAN, Manhattan Beach, CA (US); Ashley A. VO, Northridge, CA (US); Noriko AMMERMAN, Los Angeles, CA (US)

(73) Assignee: CEDARS-SINAI MEDICAL CENTER, Los Angeles, CA (US)

(21) Appl. No.: 17/415,993

(22) PCT Filed: Dec. 20, 2019
(86) PCT No.: PCT/US2019/068103

§ 371 Date: Jun. 18, 2021

Related U.S. Application Data
(60) Provisional application No. 62/783,136, filed on Dec. 20, 2018.
(60) Provisional application No. 62/855,993, filed on Jun. 1, 2019.

(51) Int. Cl. C07K 16/24; A61K 31/522; A61K 39/395; A61K 31/4196; A61K 31/505; A61K 31/635; A61P 13/12

(52) U.S. Cl. CPC C07K 16/248; A61K 31/522; A61K 39/3955; A61K 31/4196; A61K 2039/505; A61K 31/635; A61K 39/39516; A61P 13/12; C07K 2317/565; A61K 31/505

(57) ABSTRACT

Described herein are methods for treating antibody mediated rejection (ABMR), especially chronic active ABMR (cABMR), of transplanted organs using clazakizumab Human kidney transplant recipients with biopsy-proven cABMR, transplant glomerulopathy and who are donor-specific antibody positive showed stabilization of renal function and lowered DSA levels following clazakizumab treatment. The estimated glomerular filtration rate of the patients at six, 12 or even 18 months were stabilized, inflammatory markers of cABMR were reduced or stabilized, and inflammatory blood markers were reduced, since clazakizumab treatment.

### CROSS-REFERENCE TO RELATED APPLICATIONS

This application includes a claim of priority under 35 U.S.C. § 119(e) to U.S. provisional patent application No. 62/783,136, filed Dec. 20, 2018, and to U.S. provisional patent application No. 62/855,993, filed Jun. 1, 2019, the entireties of which are hereby incorporated by reference.

### FIELD OF INVENTION

This invention relates to clazakizumab, an antibody against interleukin 6, and its use in treating antibody-mediated rejection of organ transplant.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Extending the functional integrity of renal allografts is the primary goal of transplant medicine. Antibody mediated rejection (ABMR) is a unique, significant and often severe form of allograft rejection. The development of donor-specific antibodies (DSAs) post-transplantation leads to chronic active antibody-mediated rejection (cABMR) and transplant glomerulopathy (TG), resulting in the majority of graft losses that occur in the United States. This reduces the quality and length of life for patients and increases cost.

The pathophysiology of ABMR indicates a primary role for antibodies, B cells, and plasma cells. As a result, intravenous immunoglobulin therapy (IVIg), rituximab, and/or plasma exchange (plasmapheresis [PLEX]) has been leveraged for the treatment of acute ABMR. Despite some success of these therapies, post-transplantation ABMR, chronic active ABMR (cABMR), and transplant glomerulopathy (TG) remain significant problems that are often unresponsive to current therapies. Data from the Deterioration in Kidney Allograft Function study showed that most graft losses in the current era of immunosuppression have evidence of cABMR with positive complement-4d protein (C4d) staining. C4d is a degradation product of the complement pathway that binds covalently to the endothelium, and it is identified as a marker of endothelial injury and hence of antibody activity. It is estimated that 5,000 allografts are lost each year in the United States, primarily from cABMR and TG. There are no approved treatments for cABMR.

ABMR is frequently seen in patients receiving inadequate immunosuppression or who are noncompliant with anti-rejection medications and those who receive human leukocyte antigen (HLA)-incompatible transplants. In addition, TG is a known consequence of persistent DSA positivity which rapidly dissipates allograft function, resulting in graft failure and return to dialysis with attendant emotional consequences for the patients and financial consequences for the health care system. Currently there is no FDA-approved therapy, and patients are often treated with combination therapies that make analysis of efficacy difficult.

There is a large unmet clinical need for new therapies to prevent and treat cABMR and TG, as they are now leading causes of chronic allograft failure. Despite best current efforts, an ABMR percentage of approximately 25-40% in even desensitized patients is still anticipated. Empirically, 80% of these episodes occur in the first 1-3 months post-transplant and can significantly impact long-term patient and graft survival. Current estimates of de novo DSA (dnDSA)-induced ABMR indicate approximately 30% of kidney transplant patients are at risk for development of ABMR, cABMR and TG. The scope of antibody-induced injury in the transplant population is significant and growing. Recent data indicated long-term outcomes of patients with cABMR and TG are very poor. Redfield et al. evaluated graft survival in 123 patients with cABMR. Once cABMR was diagnosed, 76 patients lost their allografts with a median graft survival of 1.9 years. In addition, the graft survivals at 2 years for patients with cABMR without treatment was about 20%.

Therefore, it is an object of the present invention to provide a method of treating, mitigating or reducing the severity of ABMR and/or cABMR in patients having undergone transplantation and suffering from ABMR of the transplant, wherein the patient may be highly sensitized after the transplantation.

It is another object of the present invention to provide a method for preventing or reducing the likelihood of developing ABMR in patients in need of or having undergone an organ transplant, or preventing or reducing the likelihood of developing chronic ABMR in patients in need of an organ transplant or having undergone an organ transplant and optionally exhibiting symptoms or signs of acute antibody-mediated rejection of the organ transplant.

It is another object of the present invention to provide a method of reducing donor specific HLA antibodies and/or antibody-producing cells in patients with cABMR following organ transplantation, and/or preventing immunologic injury to the microcirculation.

### SUMMARY OF THE INVENTION

The following embodiments and aspects thereof are described and illustrated in conjunction with compositions and methods which are meant to be exemplary and illustrative, not limiting in scope.

Methods are provided for treating, inhibiting and/or reducing the severity of antibody mediated rejection (ABMR) of an organ transplant in a subject in need thereof. The methods include administering an effective amount of clazakizumab; an IL-6 binding fragment of clazakizumab; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof, in the subject. In one embodiment, the subject has undergone standard-of-care treatment for ABMR. In another embodiment, the subject's response to standard-of-care treatment is ineffective.

Also provided herein are methods for treating, inhibiting and/or reducing the severity of chronic ABMR in a kidney transplant recipient. The methods include administering an effective amount of clazakizumab; an IL-6 binding fragment of clazakizumab; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, or CDR3 or a combination thereof, which respectively are contained in SEQ ID NO: 5, 6, and 7; or a conservatively substituted, added or deleted variant thereof, in the subject.

Further provided herein are methods for reducing and/or eliminating donor specific HLA antibodies in a subject who has undergone organ transplant and is diagnosed with or exhibits symptoms of chronic ABMR and transplant glomerulopathy (TG). The methods include administering an effective amount of clazakizumab; an IL-6 binding fragment of clazakizumab; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof, in the subject.

Also provided herein are methods for treating, inhibiting and/or reducing the severity of ABMR post-organ transplant in highly HLA-sensitized patients. The methods include administering an effective amount of clazakizumab; an IL-6 binding fragment of clazakizumab; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof, in the subject to the subject, so as to treat, inhibit and/or reduce the severity of ABMR post-organ transplant in highly HLA-sensitized patients.

Further embodiments provide the clazakizumab treatment is administered sequentially or simultaneously with a standard-of-care treatment. Exemplary standard-of-care treatment includes intravenous immunoglobulin, plasmapheresis and/or rituximab.

In exemplary embodiments, the organ is one or more of heart, liver, lungs, pancreas, intestines or kidney. In one embodiment, the organ is a kidney.

In exemplary embodiments, Banff classification provides criteria for selecting, diagnosing, and/or identifying subjects for the disclosed treatment methods. Banff criteria for diagnosis of ABMR (including acute ABMR and chronic active ABMR) are detailed below. In one embodiment, a subject has ABMR or cABMR defined by Banff 2015 criteria in any of the disclosed methods. Exemplary symptoms of ABMR of kidney allograft are any one or more of: (i) deterioration of allograft function measured by serum Creatinine and estimated Glomerular filtration rate (eGFR); (ii) presence of donor-specific antibodies; (iii) biopsy evidence of capillaritis, inflammation and complement (C4d) deposition, or (iv) combinations thereof.

In some embodiments, the clazakizumab treatment is administered intravenously or subcutaneously. In exemplary embodiments, if the clazakizumab treatment is administered subcutaneously at a dose of about 20-25 mg and repeated every four weeks or monthly for at least 3, 4, 5, 6, 7, 8, 9, 10, 11, or 12 months. One embodiment provides the disclosed method includes administering six doses of 15-30 mg (or about 25 mg) each of clazakizumab at a monthly interval, followed by another six doses of 15-30 mg (or about 25 mg) each of clazakizumab at a monthly interval if estimated glomerular filtration rate (eGFR) and serum creatinine (SCr) are improved compared to index biopsy from healthy subjects or transplant recipient without symptoms of ABMR.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 is a schematic showing the treatment protocol for Example 1 to investigate the safety and efficacy of clazakizumab in treating patients with biopsy-proven cABMR, transplant glomerulopathy (TG) and DSA+ (sensitized). The study is open label, single center one-arm study that enters patients diagnosed with cABMR+TG by renal biopsy and who have eGFR of >30 cc/min at time of diagnosis. Entered patients receives subcutaneous clazakizumab 25 mg every 4 weeks (30 days) for a total of 6 doses; followed by a biopsy at the 6-month time point; and continued to have monthly clazakizumab for an additional 6 months. After 12 months of therapy, patients were able to enter a long-term extension (LTE) to receive clazakizumab 25 mg subcutaneously every other month. Patients were monitored for DSA relative intensity scores (RIS) (RIS=0 when no DSA; RIS=2 when a weak DSA level, i.e., ≤5000 MFI; RIS=5 when there is a moderate DSA level, i.e., 5000-10⁴MFI; RIS=10 when there is a strong DSA level, i.e., =>10⁴ MFI), renal function, CRP levels, and T-regulatory (Treg) cell responses. In FIG. 1, * denotes that patients with cABMR may have been treated with other therapies (e.g., pulse steroids, PLEX, and anti-CD20 (e.g., rituximab)) prior to entry into the study. All patients with previous treatments showed inadequate response to these therapies and therefore will be offered entry into this study. Patients entered into the study must have completed initial standard-of-care therapies at least one month prior to consent for clazakizumab. Patients who receive IVIG are allowed entry into study at least three days from a last dose; \hat{ }protocol biopsy to be performed on day 365 only in those who received the second round of dosing; ¹ collected on day 365 if patient receives second round of dosing; ² collected on days 0, 270, 330 and 365 if patient receives second round of dosing. Baseline is considered as Day −15.

FIG. 2 is a bar graph showing the calculated Banff scores of the eight patients in the study before and after six months of clazakizumab dosing.

FIG. 3A is a graph showing quantifications of both the summations and the average of the relative intensity scores of DSA levels of all eight patients in the study at different time points (historical baseline levels, the baseline level immediately before the study, at three months' of clazakizumab dosing, and at six months' of clazakizumab dosing). FIG. 3B is a graph showing the number of patients as having strong, moderate, weak or no DSA at various time points (historical baseline levels, the baseline level immediately before the study, at three months' of clazakizumab dosing, and at six months' of clazakizumab dosing).

FIGS. 4A-4O show the relationship of serum cytokines measured at various times post-transplant. FIGS. 4A-4E show the serum cytokines (sCr, IL-6, IL-10, IL-17A and IFN-γ, respectively) for patients who had for cause biopsies showing acute rejection (AR) (unfilled circles) vs. those who did not have biopsies of acute rejection (no AR) (filled dots). As shown, the IL-6 levels are quite low in patients with quiescent allografts. FIGS. 4F-4J show the serum cytokines (sCr, IL-6, IL-10, IL-17A and IFN-γ, respectively) for patients with antibody-mediated rejection (“AMR”, filled dots) vs. with cell-mediated rejection (“CMR”, unfilled circles.) The X axis shows time before, at and after biopsies. IL-6 levels appear to diminish with treatment of ABMR. FIGS. 4K-4O show the cytokine levels (sCr, IL-6, IL-10, IL-17A and IFN-γ, respectively) in patients who had biopsies that did not show allograft rejection (“CNI” denotes calcineurin inhibitor). The data indicates that elevations of serum IL-6 levels could be used as an early marker for allograft dysfunction mediated by antibody injury.

FIG. 5A shows representative microscopic images of the staining of normal kidney tissue (native, n=6), of transplants without rejection (tx, n=9), of tissue from a patient with cellular rejection (CMR, n=12), and of a biopsy from a patient with antibody-mediated rejection (ABMR, n=11). There are numerous IL-6+ cells in the biopsy of ABMR compared with cellular-mediated rejection and normal tissue. FIG. 5B shows data from a larger analysis of ABMR biopsies compared to other diagnoses. Morphometric scanning analysis shows a significant increase in IL-6 expression in biopsies with ABMR.

FIGS. 6A and 6B depict inflammatory cytokine & receptor levels in serum obtained pre- and post-clazakizumab (CLZ) in patients with active/chronic ABMR. FIG. 6A depicts the levels of IL-6, IL-10, interferon gamma (IFNγ) and IL-17A, and FIG. 6B depicts the levels of soluble interleukin-6 receptor (sIL-6R) and soluble gp130 (sgp130).

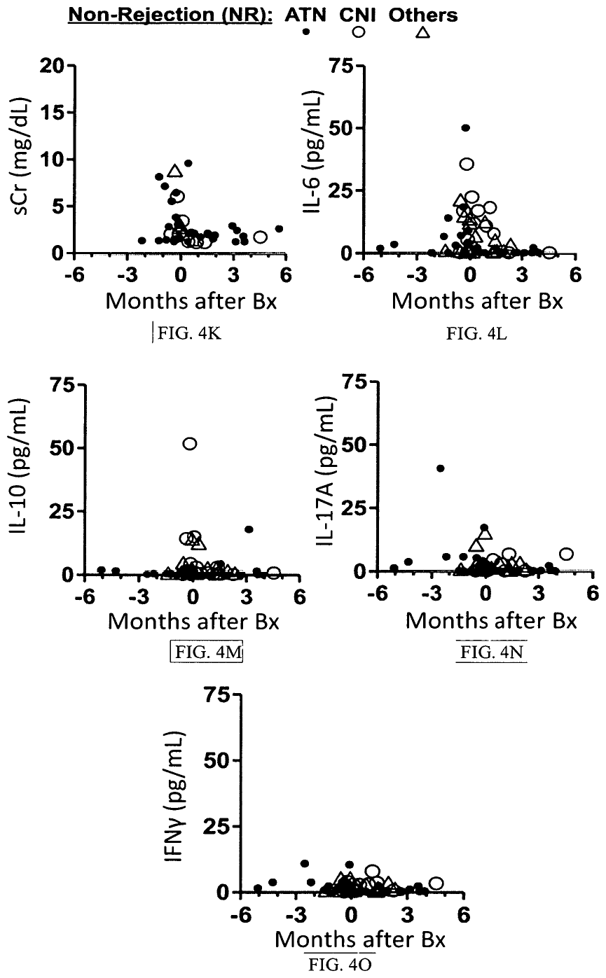

FIG. 7 depicts C-reactive protein pre- and post-clazakizumab.

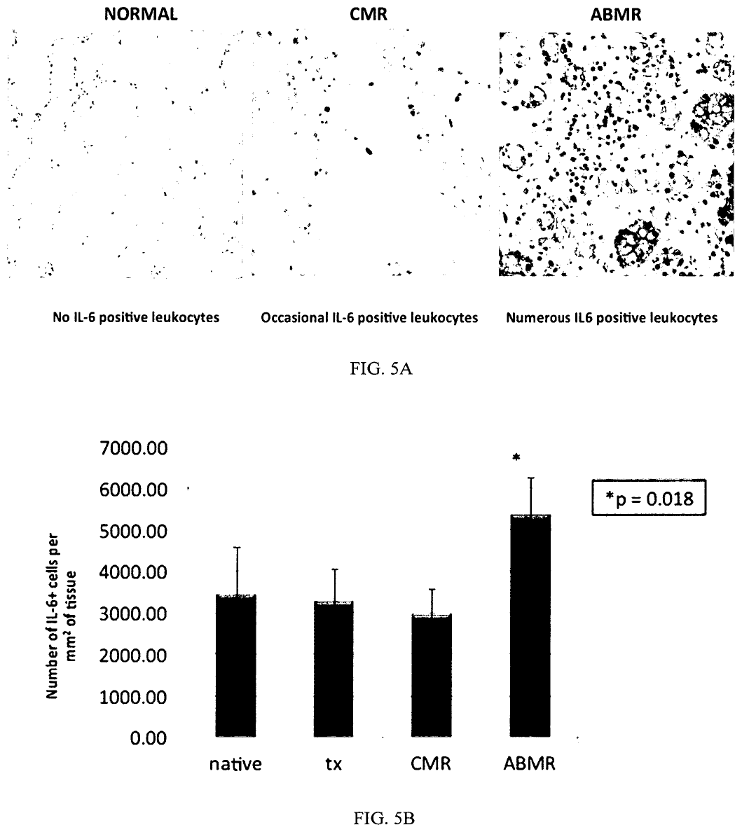

FIG. 8 depicts the levels of IgG subclasses in plasma obtained pre- and post-clazakizumab (claza) in patients with active/chronic ABMR. The levels of IgG1, IgG2, IgG3 and IgG4 in plasma obtained pre- and post-claza (0, 3 and 6M post-claza) in 8 patients with ABMR pre-claza were measured by ELISA. Of 8 patients, one patient (ShikhaleevD) received IVIG infusion right before the 1st dose of claza, the results from this patient were excluded from Ig level analysis. IgG 1 and IgG2 levels significantly decreased 6 months post-claza, while IgG3 and IgG4 levels did not significantly change. IgG3 reduction was not observed in claza-treated patients. Together with reduced total IgG levels post-claza, claza reduces IgG production likely via blockade of IL-6, B cell growth factor.

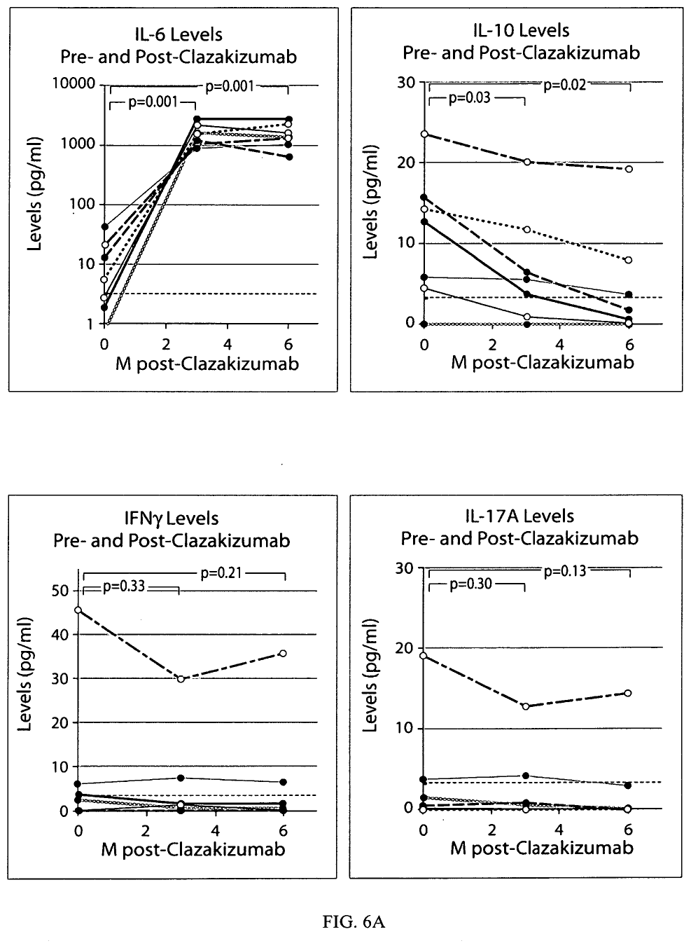

FIG. 9 depicts ABMR biopsy gene scores pre- and post-claza biopsies SH2D1B+CCL3+KLRF 1.

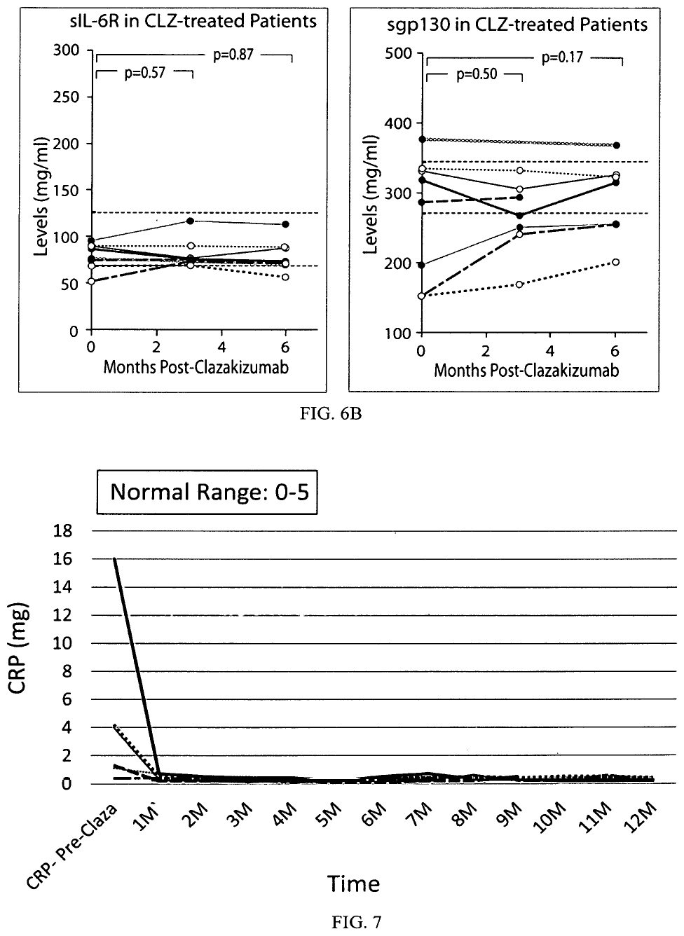

FIG. 10 depicts eGFR pre- and post-clazakizumab Treatment in cABMR Patients.

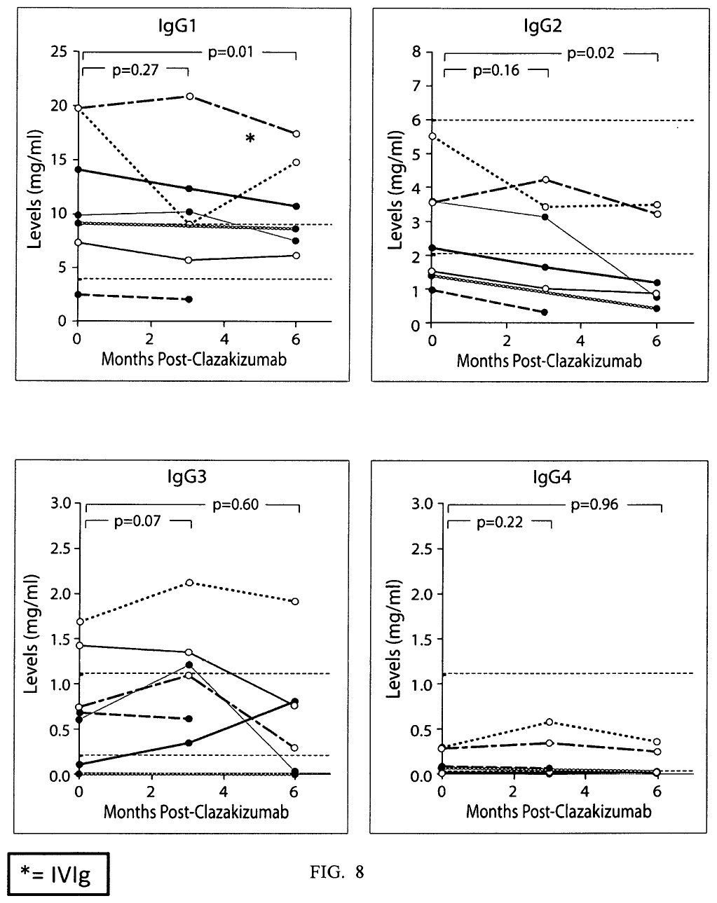

FIG. 11 depicts eGFR pre- and 12M post-clazakizumab (anti-IL-6).

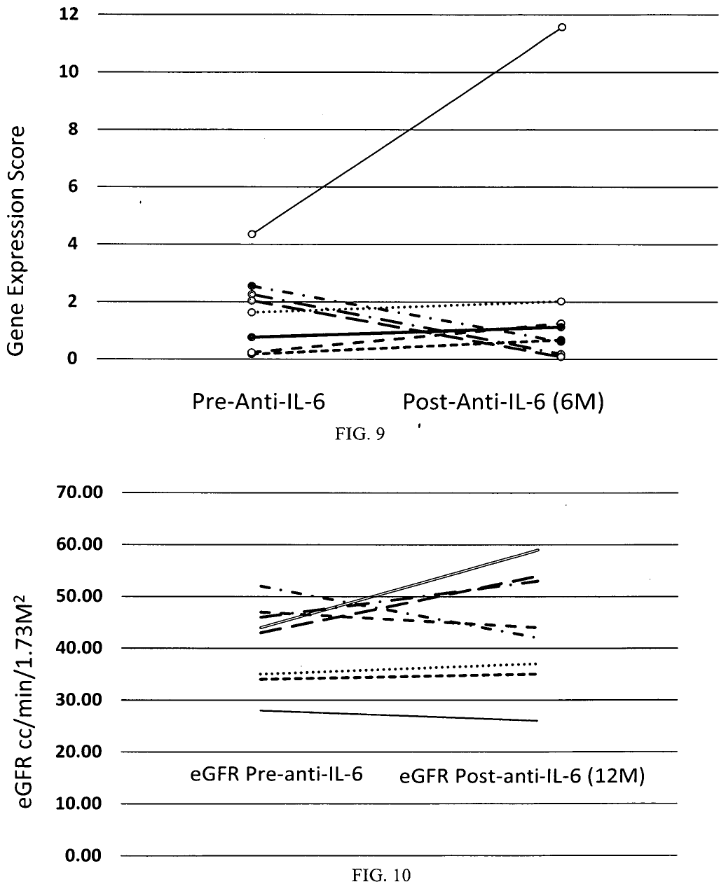

FIG. 12 shows IL-6 drives B-Cell activation and differentiation to antibody-producing plasma cells.

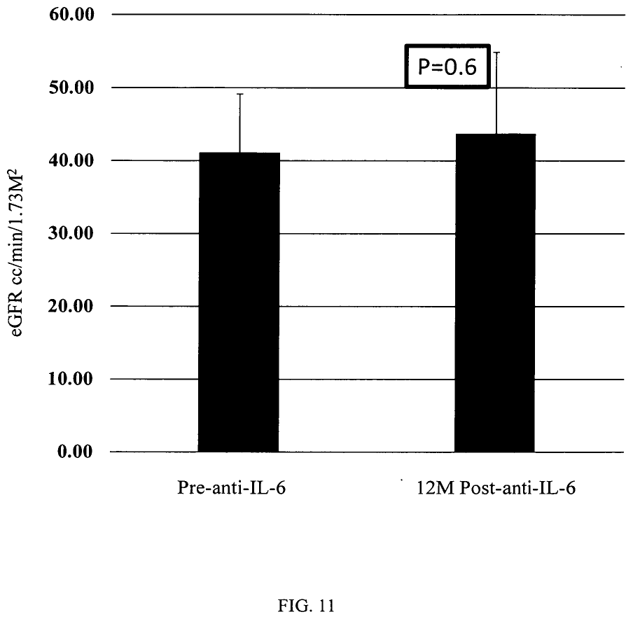

FIG. 13 depicts the level of mean fluorescence density of DSA of the patients at various time points (historical value, baseline value right before clazakizumab study, 6-month into the study, 12-month into the study, and 18-month into the study).

FIG. 14 depicts the level of eGFR of the patients at various time points (at 0-month, 6-month, 12-month and 18-month of the study).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed, Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

A “subject,” or in various embodiments “transplant recipient,” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In another embodiment, the subject is a human. In addition, the methods described herein can be used to treat domesticated animals and/or pets.

“HLA-sensitized (HS) patient” generally refers to patients whose calculated panel reactive antibodies (cPRA) or percentage of likely cross-match incompatible donors is ≥50%, who in various embodiments also has demonstrable DSA using LIMINUX bead technology and a history of sensitizing events (previous transplants, blood transfusions and/or pregnancies). The presence of HLA specific antibodies can be determined by testing patient sera against cells from a panel of HLA typed donors or against solubilized HLA antigens attached to solid supports. Generally, HLA-sensitized patients refer to patients whose cPRA is no less than 10%, 20%, 30%, 40% or 50%. In various aspects, HLA-sensitized patients refer to patients whose cPRA is no less than 50%. A positive crossmatch (+CMX) indicates the presence of donor specific alloantibodies (DSA) in the serum of a potential recipient.

Banff classification system can be used in transplant diagnostics. Transplant can be kidney, pancreas, liver, heart, lung or vascularized composite allograft, among others. For example for renal allografts, diagnostics regarding antibody-mediated rejection (ABMR), T cell-mediated rejection (TCMR), and mixed rejection can have different aspects or features. In some aspects, subjects with ABMR of transplant(s) are non-highly sensitized with de novo DSAs. In other aspects, subjects with ABMR of transplant(s) are highly sensitized. In various embodiments, non-anti-HLA DSAs can produce allograft injury alone or together with anti-HLA DSAs. Hence, in some aspects, ABMR is diagnosed based on ABMR-related pathology, namely, microcirculation inflammation, C4d deposition and vasculitis with or without increased expression of DSA-associated gene sets; and in other aspects, ABMR diagnosis based on ABMR-related pathology is accompanied by documented/evidence of DSAs.

Banff 2015 classification divides six categories, where category 1 is normal biopsy or nonspecific changes, category 2 is antibody-mediated changes—including acute/active ABMR, chronic active ABMR and C4d staining without evidence of rejection, category 3 is borderline changes including suspicious for acute TCMR, category 4 is TCMR including acute TCMR and chronic active TCMR, category 5 is interstitial fibrosis and tubular atrophy, and category 6 encompasses other changes not considered to be caused by acute or chronic rejection. According to updated Banff 2015 classification, chronic active ABMR (cABMR) includes all three features (A, B and C): A) histologic evidence of chronic tissue injury, including one or more of (a1) TG (cg>0) if no evidence of chronic thrombotic microangiopathy; includes changes evident by EM only (cg1a); (a2) severe peritubular capillary basement membrane multilayering; (a3) arterial intimal fibrosis of new onset, excluding other causes; leukocytes within the sclerotic intima favor chronic ABMR if there is no prior history of biopsy-proven TCMR with arterial involvement but are not required; B) evidence of current/recent antibody interaction with vascular endothelium, including at least one of the following: (b1) linear C4d staining in peritubular capillaries; (b2) at least moderate microvascular inflammation ([g+ptc]≥2), although in the presence of acute TCMR, borderline infiltrate or infection, ptc≥2 alone is not sufficient and g must be ≥1; (b3) increased expression of gene transcripts in the biopsy tissue indicative of endothelial injury; and C) serologic evidence of DSAs (HLA or other antigens). Generally biopsies suspicious for ABMR on the basis of meeting criteria A and B should prompt expedited DSA testing. According to updated Banff 2015 classification, acute/active ABMR has all three features (D, E and F): D) histologic evidence of acute tissue injury, including one or more of the following: (d1) microvascular inflammation (g>0 in the absence of recurrent or de novo glomerulonephritis, and/or ptc>0); (d2) intimal or transmural arteritis (v>0); (d3) acute thrombotic microangiopathy in the absence of any other cause; (d4) acute tubular injury in the absence of any other apparent cause; E) evidence of current/recent antibody interaction with vascular endothelium, including at least one of the following: (e1) linear C4d staining in peritubular capillaries; (e2) at least moderate microvascular inflammation ([g+ptc]≥2), although in the presence of acute TCMR, borderline infiltrate or infection; ptc≥2 alone is not sufficient, and g must be ≥1, (e3) increased expression of gene transcripts in the biopsy tissue indicative of endothelial injury; and F) serologic evidence of DSAs (HLA or other antigens). “cg”: glomerular double contours; “ci”: interstitial fibrosis; “ct”: tubular atrophy; “cv”: vascular fibrous intimal thickening; “g”: glomerulitis; “i”: inflammation; “ptc”: peritubular capillaritis; “t”: tubulitis; “ti”: total inflammation; “v”: intimal arteritis. Admittedly Banff criteria may update from year to year, the methods herein can be used on patients as disclosed herein where ABMR is diagnosed per contemporary Banff standard.

Transplant glomerulopathy (TG) is a morphologic lesion, featured by reduplication/multilammination of glomerular basement membrane by light microscopy or electron microscopy in the absence of immune complex deposits. Transplant glomerulopathy is a morphologic description of histologic or ultrastructural alterations. Multiple pathophysiologic mechanisms result in development of this lesion, all related to chronic, repeated endothelial cell injury.

The terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as weight loss or muscle loss resulting from cancer cachexia. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The term “administering,” refers to the placement an agent as disclosed herein into a subject by a method or route which results in at least partial localization of the agents at a desired site.

The term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, 0, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g. papain cleavage) or via chemical cleavage of intact antibodies. An antibody can be a chimeric, humanized or human antibody. An antibody can be an IgG1, IgG2, IgG3 or IgG4 antibody. In some aspects, an antibody herein has an Fc region that has been modified to alter at least one of effector function, half-life, proteolysis, or glycosylation.

The term “antibody fragment,” refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and CH1 domains; (ii) the Fab′ fragment, which is a Fab fragment having one or more cysteine residues at the C-terminus of the CH1 domain; (iii) the Fd fragment having V_{H} and CH1 domains; (iv) the Fd′ fragment having V_{H} and CH1 domains and one or more cysteine residues at the C-terminus of the CH1 domain; (v) the Fv fragment having the V_{L} and V_{H} domains of a single arm of an antibody; (vi) the dAb fragment which consists of a V_{H} domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv); (x) “diabodie” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}-CH1-V_{H}-CH1) which, together with complementary light chain polypeptides, form a pair of antigen binding regions. An antibody or antibody fragment can be scFvs, camelbodies, nanobodies, IgNAR (single-chain antibodies derived from sharks) and Fab, Fab′ or F(ab′)2 fragment.

Various aspects of disclosed methods herein include variants, derivatives or analogs of an antibody or a polypeptide. Generally, a variant of an antibody or a polypeptide contains one or more of conservative substitutions, additions and deletions. A conservative substitution, addition or deletion to a polypeptide or antibody refers to a change in the amino acid sequence compared to an original polypeptide or antibody, which results in retaining at least 80, 85, 90, 95, 96, 97, 98 or 99% identity, or at least 75%, 80%, 90%, 95%, 96%, 97%, 98%, or 99% in functional activity or binding affinity, or as much as 105%, 110%, 120%, 130%, 140%, 150%, 160%, 170%, 180%, 190%, 200%, 210%, 220%, 230%, 240%, 250%, 260%, 270%, 280%, 290% or 300% or more in functional activity or binding affinity, relative to the original antibody or polypeptide. For example, a conservatively substituted, added or deleted variant of any of SEQ ID Nos: 1-7 may result in less than 4, 3, 2 or 1 amino acid difference in identity, and/or result in at least 75%, 80%, 90%, 95%, 96%, 97%, 98%, or 99% in functional activity or binding affinity (or as much as 105%, 110%, 120%, 130%, 140%, 150%, 160%, 170%, 180%, 190%, 200%, 210%, 220%, 230%, 240%, 250%, 260%, 270%, 280%, 290% or 300% or more) of the functional activity or binding affinity to IL-6 compared with the original sequence.

“Selectively binds” or “specifically binds” refers to the ability of an antibody or antibody fragment thereof described herein to bind to a target, such as a molecule present on the cell-surface, with a K_{D} 10⁻⁵M (10000 nM) or less, e.g., 10⁻⁶ M, 10⁻⁷M, 10⁻⁸M, 10⁻⁹M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

“Ineffective” treatment refers to when a subject is administered a treatment and there is less than 5%, improvement in symptoms. If specifically provided for in the claim, ineffective treatment can refer to less than 1%, 2%, 3%, 4%, 6%, 7%, 8%, 9% or 10% improvement in symptoms.

“Adverse Events,” an adverse event is any unfavorable and unintended sign, symptom, or disease temporally associated with the use of an investigational medicinal product (IMP) or other protocol-imposed intervention, regardless of attribution. An adverse event can be any unfavorable and unintended sign (including an abnormal laboratory finding), symptom, or disease temporally associated with the use of a medicinal product, whether or not considered related to the medicinal product. Surgical procedures are not adverse events; they are therapeutic measures for conditions that require surgery. However, the condition for which the surgery is required is an adverse event, if it occurs or is detected during the Study as shown in the Example. Planned surgical measures and the condition(s) leading to these measures are not adverse events, if the condition(s) was (were) known before the start of Study treatment. In the latter case, the condition should be reported as medical history. Adverse events (AEs) include (1) AEs not previously observed in the subject that emerge during the protocol-specified AE reporting period, including signs or symptoms associated with Clazakizumab infusion that were not present prior to the AE reporting period; (2) complications that occur as a result of protocol-mandated interventions (e.g., renal protocol biopsy); (3) if applicable, AEs that occur prior to assignment of study treatment associated with medication washout, no treatment run-in, or other protocol-mandated intervention; (4) preexisting medical conditions (other than the condition being studied) judged by the investigator to have worsened in severity or frequency or changed in character during the protocol-specified AE reporting period.

An adverse event should be classified as a serious adverse event (SAE) if the following criteria are met: (1) it results in death (i.e., the AE actually causes or leads to death); (2) it is life threatening (i.e., the AE, in the view of the investigator, places the subject at immediate risk of death. It does not include an AE that, had it occurred in a more severe form, might have caused death); (3) it requires or prolongs inpatient hospitalization; (4) it results in persistent or significant disability/incapacity (i.e., the AE results in substantial disruption of the subject's ability to conduct normal life functions); (5) it results in a congenital anomaly/birth defect in a neonate/infant born to a mother exposed to the IMP; or (6) it is considered a significant medical event by the investigator based on medical judgment (e.g., may jeopardize the subject or may require medical/surgical intervention to prevent one of the outcomes listed above).

“Preexisting Condition,” a preexisting condition is one that is present at the start of the Study. Preexisting conditions that worsen during the study are considered adverse events. A preexisting condition should be recorded as an adverse event if the frequency, intensity, or the character of the condition worsens during the Study period.

“Abnormal Test Findings,” an abnormal test finding that meets any one of the criteria below should be considered an adverse event:

Test result is associated with accompanying symptoms;

Test result requires additional diagnostic testing or medical/surgical intervention;

Test result leads to a change in Study treatment dosing (e.g., dose modification, interruption, or permanent discontinuation) or concomitant drug treatment (e.g., addition, interruption, or discontinuation) or any other change in a concomitant medication or therapy;

Test result leads to any of the outcomes included in the definition of a serious adverse event (note: this would be reported as a serious adverse event);

Test result is considered an adverse event by the Investigator.

Laboratory results that fall outside the reference range and do not meet one of the criteria above should not be reported as adverse events. Repeating an abnormal test, in the absence of the above conditions, does not constitute as adverse event. Any abnormal test result that is determined to be an error does not require reporting as an adverse event.

The term “statistically significant” or “significantly” refers to statistical evidence that there is a difference. It is defined as the probability of making a decision to reject the null hypothesis when the null hypothesis is actually true. The decision is often made using the p-value.

Clazakizumab and Methods of Use

Methods are provided for treating, reducing severity, or improving transplant outcomes in a patient diagnosed with or exhibiting signs of acute ABMR, chronic active ABMR, or chronic active ABMR with TG, wherein the patient in some embodiments is highly-sensitized and in other embodiments is non-sensitized. The methods in various embodiments include administering to the subject an effective amount of clazakizumab, or an antibody or antigen-binding fragment thereof which shares at least 75%, 76%, 77%, 78%, 79%, 80%, 81%, 82%, 83%, 84%, 85%, 86%, 87%, 88%, 89%, 90%, 91%, 92%, 93% 94%, 95%, 96%, 97%, 98% or 99% sequence homology (identical) to clazakizumab or the complementarity-determining regions (CDRs) of clazakizumab, or which retains at least 75%, 80%, 90%, 95%, 96%, 97%, 98%, or 99% of the binding capability to IL-6 or of the functional activity. compared to clazakizumab or a complementarity-determining region (CDR) thereof.

Methods for reducing donor specific antibodies in transplant recipients diagnosed with or showing signs of acute ABMR, chronic active ABMR, or chronic active ABMR and TG are also provided, including administering to the subject an effective amount of clazakizumab, or an antibody or antigen-binding fragment thereof which shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence homology to clazakizumab or the complementarity-determining regions (CDRs) of clazakizumab, or which retains at least 75%, 80%, 90%, 95%, 96%, 97%, 98%, or 99% of the binding capability to IL-6 or of the functional activity. compared to clazakizumab or a complementarity-determining region (CDR) thereof.

Clazakizumab is a glycosylated humanized (from a rabbit parental antibody) monoclonal antibody targeting interleukin-6. The peptide sequence and structural information of clazakizumab are available from IMGT/mAb-db record #414. BLAST peptide sequence analysis reveals identical matches with peptides claimed in U.S. Pat. No. 8,062,864, which is herein incorporated by reference in its entirety. Further description of clazakizumab and its variants is shown in U.S. Pat. No. 7,935,340, which is herein incorporated by reference in its entirety, whose antibodies or antibody fragments are in some embodiments used in the methods disclosed herein for treating ABMR or cABMR in a subject in need of or having undergone allograft transplantation. For example, clazakizumab or an antibody or antibody fragment for use in the disclosed methods has V_{H} polypeptide containing CDR1, CDR2, and CDR3 polypeptides which respectively are contained in SEQ ID NO: 1 (for CDR1 of V_{H}), SEQ ID NO: 2 or SEQ ID NO:3 (for CDR 2 of V_{H}), SEQ ID NO: 4 (for CDR3 of V_{H}), and has V_{L} polypeptide containing CDR1, CDR2, and CDR3 polypeptides which respectively are contained in SEQ ID NO: 5, 6, and 7. The anti-human IL-6 antibody includes a variable heavy chain contained in SEQ ID NO: 8, 9 or 10, and a variable light chain contained in SEQ ID NO: 11 or 12.

(SEQ ID NO: 1)
Asn-Tyr-Tyr-Val-Thr
(SEQ ID NO: 2)
Ile-Ile-Tyr-Gly-Ser-Asp-Glu-Thr-Ala-
Tyr-Ala-Thr-Trp-Ala-Ile-Gly
(SEQ ID NO: 3)
Ile-Ile-Tyr-Gly-Ser-Asp-Glu-Thr-Ala-Tyr-
Ala-Thr-Ser-Ala-Ile-Gly
(SEQ ID NO: 4)
Asp-Asp-Ser-Ser-Asp-Trp-Asp-Ala-Lys-Phe-Asn-Leu
(SEQ ID NO: 5)
Gln-Ala-Ser-Gln-Ser-Ile-Asn-Asn-Glu-Leu-Ser
(SEQ ID NO: 6)
Arg-Ala-Ser-Thr-Leu-Ala-Ser
(SEQ ID NO: 7)
Gln-Gln-Gly-Tyr-Ser-Leu-Arg-Asn-Ile-Asp-Asn-Ala.
A variable heavy chain sequence is
set forth in SEQ ID NO: 8 -
METGLRWLLLVAVLKGVQCQSLEESGGRLVTPGTPLTLTCTASGFSLS
NYYVTWVRQAPGKGLEWIGIIYGSDETAYATWAIGRFTISKTSTTVDL
KMTSLTAADTATYFCARDDSSDWDAKFNLWGQGTLVTVSSASTKGPSV
FPLAPSSKSTSGGTAALGCLVK.
A substituted variable heavy chain sequence
is set forth in SEQ ID NO: 9 -
EVQLVESGGGLVQPGGSLRLSCAASGFSLSNYYVTWVRQAPGKGLEWVG
IIYGSDETAYATWAIGRFTISRDNSKNTLYLQMNSLRAEDTAVYYCARD
DSSDWDAKFNL.
Another substituted variable heavy chain
sequence is set forth in SEQ ID NO: 10 -
EVQLVESGGGLVQPGGSLRLSCAASGFSLSNYYVTWVRQAPGKGLEW
VGIIYGSDETAYATSAIGRFTISRDNSKNTLYLQMNSLRAEDTAVYY
CARDDSSDWDAKFNL.
A variable light chain sequence is
set forth in SEQ ID NO: 11 -
MDTRAPTQLLGLLLLWLPGARCAYDMTQTPASVSAAVGGTVTIKCQASQ
SINNELSWYQQKPGQRPKWYRASTLASGVSSRFKGSGSGTEFTLTISDL
ECADAATYYCQQGYSLRNIDNAFGGGTEVVVKRTVAAPSVFIFPPSDEQ
LKSGTASVVCLLNN.
A substituted variable light chain sequence
is set forth in SEQ ID NO: 12 -
IQMTQSPSSLSASVGDRVTITCQASQSINNELSWYQQKPGKAP
KWYRASTLASGVPSRFSGSGSGTDFTLTISSLQPDDFATYYCQ
QGYSLRNIDNA.

Clazakizumab is a genetically engineered humanized immunoglobulin G1 (IgG1) antibody that binds to human IL-6 with an affinity of 4 pM. Using multiple assays for signaling and cellular functions in response to IL-6 alone (to measure classical signaling) and a combination of IL-6 and sIL-6R (to measure trans-signaling), it was demonstrated that clazakizumab is a potent and full antagonist of IL-6-induced signaling as measured by phosphorylation of signal transducer and activator of transcription 3 (STAT3), as well as cellular functions such as cell proliferation, differentiation, activation, B-cell production of immunoglobulins, and hepatocyte production of acute phase proteins (C-reactive protein [CRP] and fibrinogen). In addition, clazakizumab is shown to be a competitive antagonist of IL-6-induced cell proliferation. Although clazakizumab has been evaluated in patients with rheumatoid arthritis, it has not yet been approved by the FDA for any condition. Before Applicant's invention, there was no information for clazakizumab in HS patients awaiting incompatible (HLAi) transplants or for treatment of antibody-mediated rejection.

IL-6 is a key cytokine that regulates inflammation and the development, maturation, and activation of T cells, B cells, and plasma cells. Excessive IL-6 production has been linked to a number of human diseases characterized by unregulated antibody production and autoimmunity. IL-6/IL-6R interactions are important for alloantibody generation as shown in an animal model of alloimmunity. Blockade of these interactions with an anti-IL-6R monoclonal results in significant reductions of alloantibodies, antibody production by splenic and bone marrow plasma cells, direct inhibition of plasma cell anti-HLA antibody production and induction of T_{reg} cells with inhibition of T-follicular (T_{fh}) cells. Thus, IL-6 shapes T-cell immunity and is a powerful stimulant for pathogenic IgG production.

Various embodiments provide methods for reducing donor-specific antibodies (e.g., donor specific HLA antibodies) and treating or reducing the severity of chronic ABMR, chronic ABMR in combination with TG, or acute ABMR of organ transplant in a subject, where the method includes administering an effective amount of clazakizumab; antigen-binding fragment thereof; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7, to the subject. Some embodiments of these methods provide further selecting a subject that is highly sensitized, as characterized by having calculated panel reactive antibodies (cPRA) or percentage of likely cross-match incompatible donors of at least 10%, 20%, 30%, 40%, 50%, 60%, or 70%. In one embodiment, the methods further include selecting a subject that is highly sensitized, characterized by having cPRA or percentage of likely cross-match incompatible donors of at least 50%. Another embodiment of the invention provides a method for reducing donor-specific antibodies (e.g., donor specific HLA antibodies) and treating or reducing the severity of chronic ABMR, chronic ABMR in combination with TG, or acute ABMR of transplanted allograft(s) in a subject involves administering an antibody or a polypeptide, which is capable of binding to IL-6 and which contains one or more amino acid sequences that include conservative substitutions, additions and/or deletions to the amino acid sequence in one or more CDRs of clazakizumab or in one or more of amino acid sequences set forth in SEQ ID Nos: 1-7. In one aspect, the conservative substitutions, additions and/or deletions result in less than 4, 3, 2 or 1 amino acid difference in identity to a CDR of clazakizumab or to a CDR set forth in any one of SEQ ID Nos: 1-7. In another aspect, the conservative substitutions, additions and/or deletions result in 80, 85, 90, 95, 96, 97, 98 or 99% identity/homology to a CDR of clazakizumab or to a CDR set forth in any one of SEQ ID Nos: 1-7. Yet another aspect provides the conservative substitutions, additions and/or deletions result in at least 75%, 80%, 90%, 95%, 96%, 97%, 98%, or 99% of the binding capability to IL-6 of clazakizumab or of the functional activity of clazakizumab. An aspect provides the conservative substitutions, additions and/or deletions confer 105%, 110%, 120%, 130%, 140%, 150%, 160%, 170%, 180%, 190%, 200%, 210%, 220%, 230%, 240%, 250%, 260%, 270%, 280%, 290% or 300% or more of the binding capability to IL-6 compared to that of clazakizumab or of the functional activity of clazakizumab. Further embodiments of these methods provide administering an effective amount of an anti-human IL-6 antibody or antibody fragment which includes a variable heavy chain in SEQ ID NO: 8, 9 or 10 and a variable light chain in SEQ ID NO: 11 or 12 to a subject in need thereof or diagnosed with or exhibiting signs of chronic ABMR, chronic ABMR in combination with TG, or acute ABMR.

Various embodiments provide a method for treating or reducing the severity of ABMR post-kidney transplantation, and optionally reducing donor-specific HLA antibodies, in a human subject, where the method includes administering an effective amount of IVIG and an effective amount of clazakizumab; an IL-6 binding fragment of clazakizumab; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7. Another embodiment of the invention provides a method for treating or reducing the severity of ABMR post-kidney transplantation, and optionally reducing donor-specific HLA antibodies, in a human subject involves administering an antibody or a polypeptide, which is capable of binding to IL-6 and which contains one or more amino acid sequences that include conservative substitutions, additions and/or deletions to the amino acid sequence in one or more CDRs of clazakizumab or in one or more of amino acid sequences set forth in SEQ ID Nos: 1-7. In one aspect, the conservative substitutions, additions and/or deletions result in less than 4, 3, 2 or 1 amino acid difference in identity to a CDR of clazakizumab or to a CDR set forth in any one of SEQ ID Nos: 1-7. In another aspect, the conservative substitutions, additions and/or deletions result in 80, 85, 90, 95, 96, 97, 98 or 99% identity/homology to a CDR of clazakizumab or to a CDR set forth in any one of SEQ ID Nos: 1-7. Yet another aspect provides the conservative substitutions, additions and/or deletions result in at least 75%, 80%, 90%, 95%, 96%, 97%, 98%, or 99% of the binding capability to IL-6 of clazakizumab or of the functional activity of clazakizumab. An aspect provides the conservative substitutions, additions and/or deletions confer 105%, 110%, 120%, 130%, 140%, 150%, 160%, 170%, 180%, 190%, 200%, 210%, 220%, 230%, 240%, 250%, 260%, 270%, 280%, 290% or 300% or more of the binding capability to IL-6 or of the functional activity of clazakizumab.

In one embodiment, IVIG is administered prior to clazakizumab; an IL-6 binding fragment of clazakizumab; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof.

Various embodiments provide a method for treating or reducing the severity of ABMR post-organ transplantation, and optionally reducing donor-specific HLA antibodies, in a human subject, where the method includes administering an effective amount of a combination of IVIG and clazakizumab; an effective amount of the combination of IVIG and an IL-6-binding fragment of clazakizumab; or an effective amount of the combination of IVIG and a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof. In one embodiment, IVIG is administered prior to or concurrent with clazakizumab; an IL-6 binding fragment of clazakizumab; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof.

Yet more embodiments provide a method for reducing donor-specific HLA antibodies in a transplant recipient exhibiting symptoms of chronic ABMR, where the method includes administering an effective amount of a combination of IVIG, plasmapheresis and clazakizumab; an effective amount of the combination of IVIG, plasmapheresis and an IL-6-binding fragment of clazakizumab; or an effective amount of the combination of IVIG, plasmapheresis and a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof. In one embodiment, IVIG and plasmapheresis are administered prior to or concurrent with clazakizumab; an IL-6 binding fragment of clazakizumab; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof.

As seen in Examples below, patients diagnosed with cABMR and TG after being treated with clazakizumab showed stabilization of renal function and improvements in DSA relative intensity scores (e.g., significantly reduced amount of DSA compared to before clazakizumab treatment). Further, biopsy findings showed trends in reduced g+ptc, cg and C4d scores. In some aspects of the methods, pre- and post-biopsy molecular microscope analysis showed stabilization and sharp reductions in patients. In some aspects, the disclosed methods do not induce serious adverse events in the patients. In some aspects, the disclosed methods resulted in reductions in IgG3 level in the patients. In some aspects, the disclosed methods include that after about 6, 7, 8, 9, 10, 11, 12 or more months of dosing clazakizumab or an antigen-binding fragment thereof, the subject has an increased level of regulatory T cells. Further aspects of the disclosed methods include that the function of allograft kidney in subjects after treatment with clazakizumab or antigen-binding fragments thereof are stabilized, characterized by comparable levels of estimated glomerular filtration rate across 3 months, 6 months, 12 months or even 18 months post initial dosing of the clazakizumab or the antigen-binding fragments thereof.

Patient Populations

Allograft rejection can be hyperacute (occurring within minutes after the vascular anastomosis), acute (occurring days to weeks after transplantation), late acute (occurring 3 months after transplantation), or chronic (occurring months to years after transplantation)

Various embodiments of the methods provide further steps of diagnosing and/or selecting a subject with ABMR, which often is measured by the Banff classification, electronic microscopic examination of biopsies, an antigen-based bead assay, or a combination thereof. Other embodiments provide the method further includes diagnosing and/or selecting a subject exhibiting symptoms of ABMR (e.g., chronic active ABMR) and transplant glomerulopathy, before administering an effective amount of clazakizumab. An aspect of the disclosed method provides diagnosing or selecting a subject that is diagnosed with the presence of anti-HLA antibodies via a single-antigen bead testing. Another aspect of the disclosed method provides diagnosing or selecting a transplant recipient whose biopsies are examined to exhibit allograft rejection under electron microscopy.

Various embodiments of the methods include that the subject is diagnosed with or exhibiting signs of chronic active ABMR of an allograft before the administration of clazakizumab or an antigen-binding fragment thereof. Some embodiments of the methods further include identifying or selecting a subject diagnosed with or exhibiting signs of chronic active ABMR of an allograft, and administering to the subject an effective amount of clazakizumab, an antigen-binding fragment thereof, or an antibody or antibody fragment disclosed above. One embodiment provides a method of treating or reducing the severity of chronic antibody-mediated rejection in a transplant recipient, which includes administering to the recipient an effective amount of clazakizumab; an IL-6 binding fragment of clazakizumab; or a disclosed polypeptide, wherein the recipient is diagnosed or exhibits at least features A, B and C: (A) histologic evidence of chronic tissue injury, defined by the presence of at least one of the following: (i) transplant glomerulopathy (cg>0) in the absence of chronic TMA; (ii) severe peritubular capillary basement membrane multilayering identified by electron microscopy; and (iii) new-onset arterial intimal fibrosis with no other known etiology; (B) histologic evidence of antibody interaction with vascular endothelium, defined by the presence of at least one of the following: (i) linear C4d staining in the peritubular capillaries; (ii) at least moderate microvascular inflammation (g+ptc>2); and (iii) increased expression of tissue gene transcripts indicative of endothelial injury; (C) detectable DSAs (HLA or non-HLA) in the serum. Another embodiment provides a method of treating or reducing the severity of chronic antibody-mediated rejection in a transplant recipient, which includes administering to the recipient an effective amount of clazakizumab; an IL-6 binding fragment of clazakizumab; or a disclosed polypeptide, following selecting a recipient that is diagnosed or exhibits at least features A, B and C: (A) histologic evidence of chronic tissue injury, defined by the presence of at least one of the following: (i) transplant glomerulopathy (cg>0) in the absence of chronic TMA; (ii) severe peritubular capillary basement membrane multilayering identified by electron microscopy; and (iii) new-onset arterial intimal fibrosis with no other known etiology; (B) histologic evidence of antibody interaction with vascular endothelium, defined by the presence of at least one of the following: (i) linear C4d staining in the peritubular capillaries; (ii) at least moderate microvascular inflammation (g+ptc>2); and (iii) increased expression of tissue gene transcripts indicative of endothelial injury; (C) detectable DSAs (HLA or non-HLA) in the serum.

Various embodiments of the methods include that the subject is diagnosed with or exhibiting signs of acute ABMR of an allograft before the administration of clazakizumab or an antigen-binding fragment thereof. Some embodiments include identifying or selecting a subject diagnosed with or exhibiting signs of acute ABMR of an allograft, and administering to the subject an effective amount of clazakizumab, an antigen-binding fragment thereof, or an antibody or antibody fragment disclosed above.

Some embodiments of the methods include that the subject is diagnosed with chronic active ABMR and exhibiting TG before the administration of clazakizumab or an antigen-binding fragment thereof. Some embodiments of the methods include identifying or selecting a subject diagnosed with chronic active ABMR and exhibiting TG, and administering to the subject an effective amount of clazakizumab, an antigen-binding fragment thereof, or an antibody or antibody fragment disclosed above.

Some embodiments of the methods include that the subject is diagnosed with chronic active ABMR of an allograft, exhibiting TG in biopsy of the allograft transplant, and are highly sensitized and/or containing DSA, before the administration of the clazakizumab or an antigen-binding fragment thereof. In various aspects, highly sensitized subject is characterized by having calculated panel reactive antibodies (cPRA) or percentage of likely cross-match incompatible donors of ≥50%. Further embodiments include identifying or selecting diagnosed with chronic active ABMR of an allograft, exhibiting TG in biopsy of the allograft transplant, and are highly sensitized and/or containing DSA, and administering to the subject an effective amount of clazakizumab, an antigen-binding fragment thereof, or an antibody or antibody fragment disclosed above.

Yet another embodiment of the methods provides that any of the subjects above has undergone other therapies including pulse steroids, PLEX, and/or anti-CD20 therapy (e.g., rituximab), and their response to these other therapies was ineffective, before the administration of clazakizumab or an antigen-binding fragment thereof. An embodiment of the methods includes identifying or selecting a subject diagnosed with ABMR and having undergone therapies such as steroids, PLEX, and/or anti-CD20 therapy, but whose response was ineffective, and administering to the subject an effective amount of clazakizumab, an antigen-binding fragment thereof, or an antibody or antibody fragment disclosed above.

Further aspects of the methods include that the subject does not have rheumatoid arthritis (RA), psoriatic arthritis (PsA), Crohn's disease, graft-versus-host disease (GVHD), a cancer, or a combination thereof. Additional aspects of the methods further include selecting a subject that does not have or has not had rheumatoid arthritis (RA), psoriatic arthritis (PsA), Crohn's disease, graft-versus-host disease (GVHD) or a cancer and that is HLA-sensitized and in need of or having undergone a solid organ (e.g., kidney) transplantation, for the methods of reducing and/or eliminating donor specific antibodies.

Various embodiments provide methods for treating or reducing the severity of ABMR of an allograft in a transplant recipient, which include administering an effective amount of clazakizumab, an IL-6 binding fragment of clazakizumab, a polypeptide containing a variable heavy chain of SEQ ID NO: 8, 9 or 10 and a variable light chain of SEQ ID NO: 12 or 12, or a polypeptide containing a variable heavy chain with CDR1 of SEQ ID NO:1, CDR2 of SEQ ID NO: 2 or 3, and CDR3 of SEQ ID NO: 4 and a variable light chain with CDR1 of SEQ ID NO:5, CDR2 of SEQ ID NO:6 and CDR3 of SEQ ID NO: 7, in one or more doses over time, wherein (1) the subject has is diagnosed with ABMR (e.g., according to Banff 2015 criteria), in some aspects diagnosed with chronic active ABMR, (2) the subject exhibits TG in biopsy of the allograft transplant, (3) the subject is highly sensitized characterized by having a calculated panel reactive antibodies of 50% or greater, (4) the subject has a high strength of donor-specific antibodies such as determined by single antigen LUMINEX bead assay and expressed as mean fluorescence intensity (MFI) of greater than 9,000, 10,000, 11,000, 12,000 or higher for class I or class II, or (5) a combination of (1)-(4). In one aspect, the subject has one of the mentioned features. In another aspect, the subject has two of the mentioned features. In yet another aspect, the subject has three of the mentioned features. In yet another aspect, the subject has four of the mentioned features.

Another embodiment of the invention provides a method for reducing donor-specific antibodies (e.g., donor specific HLA antibodies) and treating or reducing the severity of chronic ABMR of organ transplant in a subject involves administering an antibody or a polypeptide, which is capable of binding to IL-6 and which contains one or more amino acid sequences that include conservative substitutions, additions and/or deletions to the amino acid sequence in one or more CDRs of clazakizumab or in one or more of amino acid sequences set forth in SEQ ID Nos: 1-7, wherein the subject is diagnosed with ABMR (e.g., chronic active ABMR, optionally in combination with TG and/or having DSA) before the administration of the antibody or polypeptide. In one aspect, the conservative substitutions, additions and/or deletions result in less than 4, 3, 2 or 1 amino acid difference in identity to a CDR of clazakizumab or to a CDR set forth in any one of SEQ ID Nos: 1-7. In another aspect, the conservative substitutions, additions and/or deletions result in 80, 85, 90, 95, 96, 97, 98 or 99% identity/homology to a CDR of clazakizumab or to a CDR set forth in any one of SEQ ID Nos: 1-7. Yet another aspect provides the conservative substitutions, additions and/or deletions result in at least 75%, 80%, 90%, 95%, 96%, 97%, 98%, or 99% of the binding capability to IL-6 of clazakizumab or of the functional activity of clazakizumab. An aspect provides the conservative substitutions, additions and/or deletions confer 105%, 110%, 120%, 130%, 140%, 150%, 160%, 170%, 180%, 190%, 200%, 210%, 220%, 230%, 240%, 250%, 260%, 270%, 280%, 290% or 300% or more of the binding capability to IL-6 compared to that of clazakizumab or of the functional activity of clazakizumab.

Additional Steps

Various embodiments provide one or more of the disclosed methods further include performing one or more assays for the presence or absence of infections related to cytomegalovirus, Epstein-Barr virus, polyomavirus, BK virus, JC virus, parvovirus B19, or a combination thereof with the subject before, during and/or after the administration of clazakizumab, an antigen-binding fragment of clazakizumab, or an antibody or antibody fragment disclosed above. In other embodiments, one or more of the disclosed methods are featured that the subject has no detectable amount of infection related to cytomegalovirus, Epstein-Barr virus, polyomavirus, BK virus, JC virus, parvovirus B19, or a combination thereof, before and/or after the administration of clazakizumab, an antigen-binding fragment of clazakizumab, or an antibody or antibody fragment disclosed above.

Additional embodiments of the methods disclosed herein include one or more steps of immune monitoring before and/or after the administration of clazakizumab or an antibody or antibody fragment disclosed above. In various aspects, the methods include (i) administering an effective amount of clazakizumab, an IL-6 binding fragment of clazakizumab, or a polypeptide disclosed above, in one or more doses; (ii) conducting (a) immune monitoring of the subject such as assaying the subject's blood samples to quantify Treg, Tfh, Th17, B-cell, IL-6, CRP, plasma cells, IgG levels, or a combination thereof, (b) biopsy assessment of the transplant, (c) measuring glomerular filtration rate, and/or (d) measuring amount of DSA in the subject, individually for one or more times, for example, each time following the one or more doses of the clazakizumab, the IL-6 binding fragment of clazakizumab or the polypeptide, over a period of time such as 1 month, 2 months, 3 months, 4 months, 5 months, 6 months, 7 months, 8 months, 9 months, 10 months, 11 months, 12 months, 15 months, 18 months, 24 months or longer; and optionally (iii) when (a) the immune monitoring indicates an improvement in immune reactivity such as characterized by a decreased level of CRP, Treg, Tfh, Th17, B-cell, IL-6, plasma cells, or IgG, compared to a previous immune monitoring or to a baseline measurement at or before the allograft transplantation, or a comparable level of CRP, Treg, Tfh, Th17, B-cell, IL-6, plasma cells, or IgG level, relative to a healthy subject or a subject having been desensitized, when (b) the biopsy assessment of the transplant indicates absence of cell-mediated and antibody-mediated rejection, absence or reduced evidence of allograft dysfunction (e.g., determined by C4d staining and transplant glomerulopathy, using Banff 2015 criteria), and/or improvement according to Banff 2015 criteria, (c) when glomerular filtration rate is stabilized, e.g., similar or reduced level compared to the last measurement or to prior to the transplantation, or (d) when DSA amount is stabilized, e.g., similar or reduced level compared to the last measurement or to prior to the transplantation, then discontinuing, reducing the frequency, or limiting further administration of the clazakizumab, the IL-6 binding fragment of clazakizumab, or the polypeptide; when the immune monitoring indicates that the immune reactivity such as described above has not improved or the amounts of glomerular filtration rate or DSA is not stabilized, then continuing administering the clazakizumab, the IL-6 binding fragment of clazakizumab, or the polypeptide or at an adjusted dosage; and when the biopsy assessment of the transplant indicates presence of cell-mediated and/or antibody-mediated rejection, then administering a standard-of-care treatment to treat the rejection, such as IVIG, plasmapheresis, or both. In some instances, the steps of (ii) and (iii) are repeated for one, two, three, four, five, six, seven, eight, nine or ten times, or continued as needed, or until the improvement, stabilization or even cure is observed. Some embodiments provide the administration of clazakizumab or an antibody or antibody fragment disclosed herein to the subject is continued as long as the allograft function is stabilized and/or improved, although the administration frequency of the clazakizumab or the antibody or antibody fragment can be lowered.

In some aspects of the disclosed methods, the administration of a plurality of or further doses of the clazakizumab, the IL-6 binding fragment of clazakizumab, or the polypeptide is suspended for a period of time (“break”) such as 1 week, 2 weeks, 3 weeks, 4 weeks, 2 months, and 3 months, and during/after the “break”, immune reactivity is monitored or biopsy of the allograft is assessed, and depending on results, one skilled in the art will discontinue or resume the administration of the clazakizumab, the IL-6 binding fragment of clazakizumab, or the polypeptide to further reduce or eliminate DSAs in the subject.

Dosages

The effective amount of clazakizumab for a subject may be investigated or limited based on safety evaluations. Safety evaluations include medical interviews, recording of adverse events, physical examinations, blood pressure, and laboratory measurements. Subjects are generally evaluated for adverse events (all grades), serious adverse events, and adverse events requiring study drug interruption or discontinuation at each study visit for the duration of their participation in the study.

One embodiments provide clazakizumab is administered to a subject diagnosed with or exhibiting signs of ABMR (e.g., cABMR and in combination of TG) at 25 mg subcutaneously about every 30 days for a total of six doses. A further aspect of the embodiments specifies additional doses at about the monthly interval of clazakizumab at 25 mg/each are administered. Other embodiments provide clazakizumab is administered to a transplant recipient exhibiting symptoms of cABMR at 20-30 mg subcutaneously about every 30 days for a total of at least six doses.

In one embodiment, a method for reducing donor-specific HLA antibodies in a transplant recipient exhibiting symptoms of ABMR, or treating or reducing the severity of ABMR in the transplant recipient, includes, prior to transplantation administering plasma exchange (or plasmapheresis) and/or an effective amount of IVIG (e.g., at about 2 g/kg of subject, for a maximum of 140 g), and administering an effective amount of clazakizumab (e.g., at about 20-25 mg subcutaneously, every 4 weeks for at least six doses) during and/or following transplantation.

Another embodiment provides that a method for reducing donor-specific HLA antibodies in a transplant recipient exhibiting symptoms of ABMR, or treating or reducing the severity of ABMR in the transplant recipient, includes, administering clazakizumab at a dose of 0.01-0.1 mg/kg, 0.1-0.5 mg/kg, 0.5-1 mg/kg, 1-5 mg/kg, 5-50 mg/kg, or 50-100 mg/kg, which may be repeated if the response of the transplant recipient as characterized by biopsies of rejection symptoms remain or the serum level of IL-6 maintains high compared to healthy subject or transplant recipients without rejection. One aspect of the embodiment provides clazakizumab is administered at a dose of 0.05-0.5 mg/kg, optionally repeated based on the response, and the transplant recipient is human.

In another embodiment, a method for reducing donor-specific HLA antibodies in a transplant recipient diagnosed with or exhibiting symptoms of ABMR, or treating or reducing the severity of ABMR in the transplant recipient, includes administering an effective amount of clazakizumab (e.g., at about 20-25 mg subcutaneously monthly for at least 1, 2, 3, 4, 5, 6, 7, 8, 9, 10, 11 or 12 months). In a further embodiment, this method includes discontinuing clazakizumab treatment in a transplant recipient with a maintained lowered DSA level for at least 3, 6, or 12 months after the clazakizumab treatment, compared to before the clazakizumab treatment.

Some embodiments of these methods provide assaying the biopsy from the patient, and confirming a stabilized level of glomerular filtration rate (GFR) over time (e.g., less than 10%, 20%, or 30% variations across two, three, or four consecutive biopsies) and a low level (e.g., at less than 10%, 20% or 30%) of DSA compared prior to desensitization treatment. In some embodiments when the level of GFR is not stabilized or the DSA level is high, the method further includes repeated administration of an effective amount of clazakizumab, until the level of DSA is lowered, and optionally remains as lowered for at least 1, 2, 3, 4, 5 or 6 months, compared with that prior to clazakizumab treatment.

Yet another embodiment provides a method for reducing donor-specific HLA antibodies in a transplant recipient exhibiting symptoms of ABMR, or treating or reducing the severity of ABMR in the transplant recipient, includes administering an effective amount of IVIG prior to, subsequent to, or both with administering an effective amount of clazakizumab to the subject, wherein the subject has a stabilized level of glomerular filtration rate (GFR) over time (e.g., less than 10%, 20%, or 30% variations across two, three, or four consecutive biopsies) and a low level (e.g., at less than 10%, 20% or 30%) of DSA compared prior to the clazakizumab treatment.

In some embodiments, the effective amounts of clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L}, polypeptide containing CDR1, CDR2, and CDR3 polypeptides which respectively are contained in SEQ ID NO: 5, 6, and 7, suitable for administration in the disclosed methods, can be in the range of about 10-50 μg/dose, 50-100 μg/dose, 100-150 μg/dose, 150-200 μg/dose, 100-200 μg/dose, 200-300 μg/dose, 300-400 μg/dose, 400-500 μg/dose, 500-600 μg/dose, 600-700 μg/dose, 700-800 μg/dose, 800-900 μg/dose, 900-1000 μg/dose, 1000-1100 μg/dose, 1100-1200 μg/dose, 1200-1300 μg/dose, 1300-1400 μg/dose, 1400-1500 μg/dose, 1500-1600 μg/dose, 1600-1700 μg/dose, 1700-1800 μg/dose, 1800-1900 μg/dose, 1900-2000 μg/dose, 2000-2100 μg/dose, 2100-2200 μg/dose, 2200-2300 μg/dose, 2300-2400 μg/dose, 2400-2500 μg/dose, 2500-2600 μg/dose, 2600-2700 μg/dose, 2700-2800 μg/dose, 2800-2900 μg/dose or 2900-3000 μg/dose, for a total of one, two, three, four, five, six, seven, eight, nine, ten, 11, 12, 13, 14, 15 or more doses, or as needed to continue maintaining the function of allograft (e.g., kidney transplant by maintaining eGFR) and/or to continue lowering the amount of DSA in the subject, and administered at a frequency of daily, weekly, biweekly, monthly, bimonthly, quarterly or a combination thereof.

In some embodiments, the effective amounts of clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7, suitable for administration in the disclosed methods, per unit weight of a subject in the methods above include 10-100 μg, 100-200 μg, 200-300 μg, 300-400 μg, 400-500 m, 500-600 μg, 600-700 μg, 700-800 μg, 800-900 μg, 1-5 mg, 5-10 mg, 10-20 mg, 20-30 mg, 30-40 mg, 40-50 mg, 50-60 mg, 60-70 mg, 70-80 mg, 80-90 mg, 90-100 mg, 100-200 mg, 200-300 mg, 300-400 mg, 400 mg-500 mg, 500 mg-1 g, or 1 g-10 g. Unit weight of a subject can be per kg of body weight or per subject. In one embodiment, an effective amount of clazakizumab for treating ABMR and improving/maintaining allograft function in a human subject in need thereof is about 25 mg per dose, administered at about monthly, once per two months, or other frequencies determined by a medical professional. In one embodiment, an effective amount of clazakizumab for treating ABMR and improving/maintaining allograft function in a human subject in need thereof is not 25 mg per dose administered at frequencies of about monthly or once per two months.

In further embodiments, the effective amount of clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7, suitable for administration in the disclosed methods, may be in the range of 0.01-0.05 mg/kg, 0.05-0.1 mg/kg, 0.1-1 mg/kg, 1-5 mg/kg, 5-10 mg/kg, 10-50 mg/kg, 50-100 mg/kg. In additional embodiments, the effective amount of clazakizumab, an antigen-binding fragment of clazakizumab, or a disclosed polypeptide is about 1-2 mg/kg, 2-3 mg/kg, 3-4 mg/kg, 4-5 mg/kg, 5-6 mg/kg, 6-7 mg/kg, 7-8 mg/kg, 8-9 mg/kg, 9-10 mg/kg, 10-11 mg/kg, 11-12 mg/kg, 12-13 mg/kg, 13-15 mg, 15-20 mg/kg or 20-25 mg/kg. In additional embodiments, the effective amount of the clazakizumab, an antigen-binding fragment of clazakizumab, or a disclosed polypeptide is any one or more of about 100-125 mg, 125-150 mg, 150-175 mg, 160-170 mg, 175-200 mg, 155-165 mg, 160-165 mg, 165-170 mg, 155-170 mg, or combinations thereof, which may be administered over 1, 2, 3, 4, 5, 6, 7, 8, 9, 10, 11, 12, 13, 14, 15, 16, 17, or 18 doses where some are before and others are after transplantation.

In various embodiments, the clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7, suitable for administration in the disclosed methods, is administered at any one or more of the dosages described herein at least once 1-7 times per week, 1-7 times per month, or 1-12 times per year, or one or more times as needed, for 1 month, 2 months, 3 months, 4 months, 5 months 6 months, 7 months, 8 months, 9 months, 10 months, 11 months, 12 months, 14 months, 16 months, 18 months, about 24 months, about 30 months, about 36 months or combinations thereof.

In non-clinical studies, pharmacokinetic (PK)/pharmacodynamic (PD) analyses show a single dose of clazakizumab resulted in full inhibition of IL-6 activity as measured by the inhibition of IL-6-induced phosphorylated STAT3 (pSTAT3) activity in whole blood treated ex vivo with IL-6. The results of this functional PD assay correlated with drug exposures where full inhibition of pSTAT3 activity was observed when drug levels exceeded 50 ng/mL (approximately 0.3 nM). In a tissue cross-reactivity study, tissue binding of clazakizumab was observed in multiple tissues in both human and cynomolgus monkey, generally cytoplasmic in nature, and consistent with the known expression of IL-6 by cells and tissues. Results from both single- and repeat-dose nonclinical toxicology studies of up to 6 months in cynomolgus monkeys demonstrated an acceptable safety profile for clazakizumab. In a preliminary enhanced pre- and post-natal development (ePPND) study conducted in cynomolgus monkeys, an increase in the number of monkeys with retention of the placenta at parturition was observed at clazakizumab doses of 3 mg/kg (n=2) and 30 mg/kg (n=3), corresponding to doses 11 and 110 times the planned human dose of 50 mg. There were no other safety findings of clinical concern.

Clinical studies have been conducted in healthy subjects and in the following patient populations: RA, PsA, CD, graft-versus-host disease (GVHD), and oncology. These clinical studies include a total of 1,223 subjects, of which 888 subjects were exposed to clazakizumab with doses ranging from 1 mg to 640 mg given by either intravenous (IV) or subcutaneous (SC) injection for up to 48 weeks. Following the administration of clazakizumab as a 1-hour IV infusion, the PK of clazakizumab was linear over the dose ranges of 30 mg to 640 mg in healthy subjects and 80 mg to 320 mg in subjects with RA as indicated by consistent clearance at these dose levels. The T-half of clazakizumab at all doses was very similar in healthy male subjects and in subjects with RA and was consistent with that expected for a humanized IgG1 antibody. Across the doses studied, the mean T-half of clazakizumab ranged from 19.5 to 31.0 days in healthy male subjects and from 26.4 to 30.9 days in subjects with RA. The T-half of clazakizumab after SC administration in healthy male subjects was similar to the IV administration. In a Phase 1 study comparing IV and SC dosing in healthy male subjects, the mean T-half of clazakizumab was 30.7 days after a single IV dose and, 31.1 to 33.6 days after SC administration. The bioavailability of clazakizumab after SC administration was 60% of the IV formulation. Cmax was lower and Tmax was longer for the SC administration relative to IV administration. Population PK analysis of the data from clinical studies in RA, PsA and healthy subjects have indicated that body weight affects the PK of clazakizumab such that both clearance and central volume of distribution increase with increasing body weight. Therefore, heavier subjects will have lower drug exposure compared with less heavy subjects.

In Phase 2 studies in RA and PsA, doses from 5 mg SC once every 4 weeks (Q4W) up to 320 mg IV once every 8 weeks were significantly effective with clinical response evident as early as 12 weeks post treatment. One study in RA also demonstrated that the efficacy of clazakizumab is comparable or may be better than the standard of care treatment (adalimumab+methrotrexate (MTX)) in RA. Efficacy with clazakizumab was not shown in the two Phase 2 studies in oncology (head and neck cancer and non-small cell lung cancer). Two studies were terminated prematurely due to safety concerns. A Phase 2 study in Crohn's was terminated early because of GI perforation in 3 subjects who had received clazakizumab and this indication is no longer being studied. Although these subjects had multiple confounding medical issues, and the disease itself has an inherent risk of mucosal perforation, gastrointestinal perforations were also observed during the clinical studies with tocilizumab in patients with RA. Gastrointestinal perforation is a recognized risk of anti-IL-6 mAbs. After only 3 subjects were enrolled, a study in subjects with GVHD was also prematurely terminated due to 2 subjects experiencing similar serious adverse events (SAEs) (i.e., acute renal failure) which led to death. Both subjects had severe GVHD disease at the time of death.

Overall, the safety findings from the clinical studies conducted with clazakizumab to date are consistent with the known effects of blocking the IL-6 pathway (see ACTEMRA prescribing information]). Identified risks associated with clazakizumab administration include the following: infections, liver function test (LFT) abnormalities, changes in hematology parameters (i.e., neutropenia and thrombocytopenia), dyslipidemia (i.e., hypercholesterolemia and hypertriglyceridemia), and gastrointestinal perforations.

Pharmaceutical Composition

In various embodiments, a method for treating or reducing the severity of ABMR (especially cABMR, or cABMR in combination with TG) in the transplant recipient, or reducing donor-specific HLA antibodies in a transplant recipient diagnosed with or exhibiting signs of ABMR, includes administering a pharmaceutical composition which includes (1) clazakizumab, an IL-6 binding fragment of clazakizumab; a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof, and (2) pharmaceutically acceptable excipients.

The pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to amino acids, starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In one embodiment, the disclosed methods involve administering a pharmaceutical composition which includes L-histidine, L-histidine monohydrochloride, sorbitol, polysorbate-80, and water for injection, and clazakizumab, an IL-6 binding fragment of clazakizumab, a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H} and having V_{L} polypeptide containing CDR1, CDR2, and CDR3 polypeptides which respectively are contained in SEQ ID NO: 5, 6, and 7; or a conservatively substituted, added or deleted variant thereof.

In various embodiments, the pharmaceutical compositions in the disclosed method may be formulated for delivery via any route of administration. In one embodiment, the pharmaceutical composition is administered intravenously or subcutaneously to the subject. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, sub capsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection.

The pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion. Pharmaceutically acceptable solid or liquid carriers may be added to enhance or stabilize the composition, to facilitate preparation of the composition, or to provide sustained or controlled release (or increase the half-life) of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. Emulsion carriers include liposomes, or controlled release polymeric nanoparticles known in the art. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

Anti-Infectious Agents

Various embodiments provide that the methods for treating or reducing severity of ABMR or reducing DSA in a transplant recipient diagnosed with or exhibiting symptoms of cABMR further includes administering one or more anti-infectious agents, preferably post-transplantation, as a prophylaxis or therapeutics against bacterial, viral or fungal infections.

Exemplary anti-infectious agents suitable for use in the disclosed methods include antibiotics such as aminoglycosides (e.g., amikacin, gentamicin, kanamycin, neomycin, netilmicin, streptomycin, tobramycin, paromomycin), ansamycins (e.g., geldanamycin, herbimycin), carbacephems (e.g., loracarbef), carbapenems (e.g., ertapenem, doripenem, imipenem, cilastatin, meropenem), cephalosporins (e.g., first generation: cefadroxil, cefazolin, cefalotin or cefalothin, cefalexin; second generation: cefaclor, cefamandole, cefoxitin, cefprozil, cefuroxime; third generation: cefixime, cefdinir, cefditoren, cefoperazone, cefotaxime, cefpodoxime, ceftazidime, ceftibuten, ceftizoxime, ceftriaxone; fourth generation: cefepime; fifth generation: ceftobiprole), glycopeptides (e.g., teicoplanin, vancomycin), macrolides (e.g., azithromycin, clarithromycin, dirithromycin, erythromycin, roxithromycin, troleandomycin, telithromycin, spectinomycin), monobactams (e.g., aztreonam), penicillins (e.g., amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin), antibiotic polypeptides (e.g., bacitracin, colistin, polymyxin b), quinolones (e.g., ciprofloxacin, enoxacin, gatifloxacin, levofloxacin, lomefloxacin, moxifloxacin, norfloxacin, ofloxacin, trovafloxacin), rifamycins (e.g., rifampicin or rifampin, rifabutin, rifapentine, rifaximin), sulfonamides (e.g., mafenide, prontosil, sulfacetamide, sulfamethizole, sulfanilamide, sulfasalazine, sulfisoxazole, trimethoprim, trimethoprim-sulfamethoxazole (co-trimoxazole, “tmp-smx”), and tetracyclines (e.g., demeclocycline, doxycycline, minocycline, oxytetracycline, tetracycline) as well as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin combination, and tinidazole.

Further embodiments provide the methods for treating or reducing the severity of ABMR in a transplant recipient or in a subject in need thereof include administering standard-of-care regimen including tacrolimus, mycophenolate mofetil and/or steroids, along with administering an effective amount of clazakizumab, or an antibody or antigen-binding fragment thereof.

Kits

In various embodiments, the present invention provides a kit or an article of manufacture for use with transplant recipients diagnosed with or exhibiting signs of ABMR. The kit, or article of manufacture, is an assemblage of materials or components, including clazakizumab, an IL-6 binding fragment of clazakizumab, or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; a label or package insert with instructions for use; one or more vessels as containers or a packing material; and optionally one or more diluents.

The exact nature of the components configured in the kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as reducing DSA in a subject diagnosed with or exhibiting signs of ABMR. Optionally, the kit can also contain other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of an inventive composition containing clazakizumab, an IL-6 binding fragment of clazakizumab; a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7; or a conservatively substituted, added or deleted variant thereof. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Phase I/II Trial to Evaluate the Safety and Tolerability of Clazakizumab as an Agent to Eliminate Donor Specific HLA Antibodies (DSAs) and Improve Outcomes of Patients with cABMR Post-Kidney Transplantation

Brief Study Summary

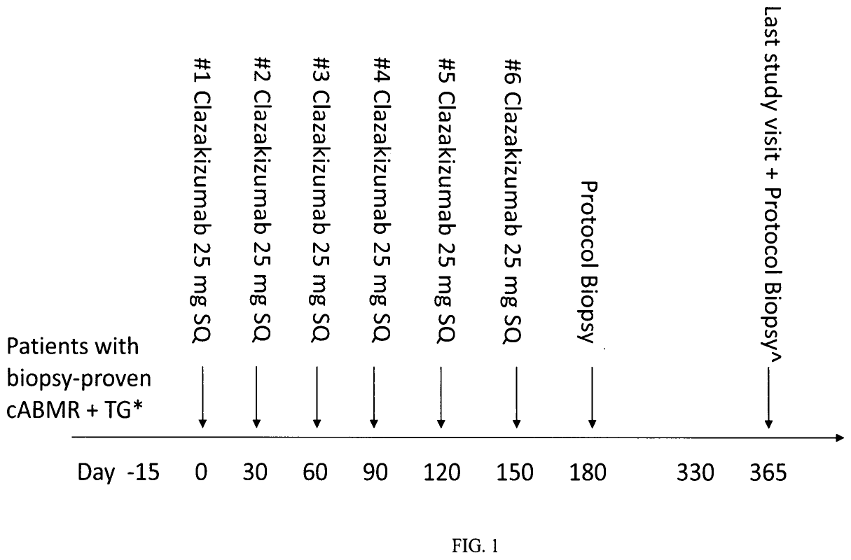

This would be a single center, phase I/II, open label single-arm exploratory study focusing on enrolling eight-ten patients with biopsy proven chronic antibody medicated rejection (cABMR), transplant glomerulopathy (TG), and donor specific antibody present (DSA+) at time of biopsy. Patients who qualify would be receiving clazakizumab (anti-IL6 monoclonal antibody) monthly×six doses. A protocol biopsy would be performed at 6 months and if improvement is seen in pathological features of cABMR from the biopsy compared to index biopsy (e.g., estimated glomerular filtration rate (eGFR), serum creatinine (SCr)), patients would continue to receive another six doses for up to 12 months. For those completing 12 doses, there would be a 12-month protocol biopsy. For those who only received six doses, the next and last study visit would be at 12 months from enrollment. Total study duration would be 12 months. (FIG. 1).

The trial would primarily examine the safety and tolerability of clazakizumab given after the diagnosis of cABMR in subjects (15-75 yrs) who exhibit DSAs to their donor. Patients entered have been diagnosed with cABMR+TG post-transplant based on Banff 2015 criteria. Patients were required to have an eGFR>30 mL/min/1.73 m² as calculated by the MDRD equation (Schwartz equation will be used to estimate CrCl for patients under 18 years of age) at entry. All patients were recruited from the renal transplant program at Cedars-Sinai Medical Center. Once cABMR was diagnosed, donor-specific anti-HLA antibodies was assessed (DSA) which were associated with cABMR and/or graft loss. DSA was detected using solid phase assay systems currently utilized at the Cedars-Sinai Medical Center HLA Laboratory. These anti-HLA antibodies may result naturally or from previous pregnancy, transfusions, or prior transplants. Patients treated with clazakizumab for cABMR would have labs for DSAs, and other monitoring labs as well as immunologic studies as outlined. In addition to the standard post-transplant immunosuppressive protocol, patients with cABMR would receive clazakizumab 25 mg SC given every 4 weeks (30 days) for a total of 6 doses. If no safety/tolerability/efficacy issues are observed after the initial dose, patients would continue the protocol as outlined. A protocol biopsy would be performed after the 6th and after the 12th doses of clazakizumab to assess the allograft for evidence of cABMR/ABMR, including C4d staining and TG using Banff 2015 criteria. Banff scoring would be compared between the index and protocol biopsy after cessation of therapy. Patients who have evidence of persistent allograft dysfunction may have non-protocol biopsies for cause. After completion of the clazakizumab therapy, patients would be followed up to assess allograft function and any cABMR episodes. In addition, several immunologic determinations would be made at time points before and after initiation of clazakizumab therapy, including assessments of T_{reg} cells (CD4+/CD25+/Fox P3+/CD127^{dim}), T helper 17 cell (Th17), T follicular helper cell (T_{fh}, CD4+, ICOS+, CXCR5+, IL-21+), circulating plasmablast (CD19+/CD38+/CD27+/IL-6+), IL-6 and C-reactive protein (CRP) levels, as well as viral PCR monitoring by center protocol for Epsten-Barr virus (EBV), cytomegalovirus (CMV), BK polyomavirus BK/JC polyomavirus, and parvovirus B19. The study includes standard-of-care maintenance regimen mainly including tacrolimus, mycophenolate mofetil and/or steroids. Patients considered for this study may have been treated with high-dose IVIG+rituximab and/or plasmapheresis; but whose response to those treatment was ineffective in terms of reducing symptoms or severity of cABMR. In addition, some patients with early, severe ABMR may have been treated with eculizumab.

The subjects were followed to determine if the use of clazakizumab for treatment of cABMR in this high-risk transplant population was safe and without infectious risks. In addition, the investigators determined the effects of clazakizumab treatment on renal biopsy assessments performed at 6 months. Assessments of renal function, donor specific antibody, and Banff 2015 biopsy scores were evaluated at that time. If improvement or stabilization observed, clazakizumab would be resumed monthly×6 doses (starting day 180 to day 330) and last study visit would be day 365 with biopsy. Study investigators would assess the transplanted patients to determine the number who sustain a viable and functioning kidney allograft as well. In the event a patient did not show improvement after receiving 6 doses of clazakizumab, no further treatment would be given and the patient would return at Day 365 for a final study visit. All subjects would be evaluated on an intent-to-treat basis. The subject accrual rate would be limited to no more than 1-2 subjects per month in the initial three months to assure safety to all subjects. Repeat laboratories would be performed at the completion of clazakizumab therapy to determine effect on levels and correlation with any potential events.

Primary Outcome Measures: Donor specific antibody elimination based on luminex HLA testing [Time Frame: 12 months]; Does clazakizumab eliminate or weaken donor specific antibodies intensities. Stabilization of clinical features of cABMR via BANFF biopsy grading criteria. [Time Frame: 12 months] Does clazakizumab help stabilize pathologic features of antibody mediated rejection at 6 month and 12 month protocol biopsies?

Secondary Outcome Measures: Serum creatinine [Time Frame: 12 months]. Serum creatinine (mg/dl) will be collected at multiple time points throughout the study to calculate eGFR. Immunologic markers [Time Frame: 12 months]. Immunologic markers collected at multiple time points throughout the study. Incidence of treatment-related adverse events [Time Frame: 12 months]. Adverse event monitoring, assessment of labs, monitoring of viral PCRs.

Inclusion Criteria: 1. Age 15-75 years at the time of screening. 2. Biopsy proven cABMR with TG on biopsy as defined by Banff 2015 and DSA positive at time of biopsy. 3. Subject/Parent/Guardian must be able to understand and provide informed consent. 4. Pneumococcal vaccinated. 5. Negative tuberculin ppd result or negative Quantiferon TB gold.

Exclusion Criteria: 1. Multi-organ transplant (e.g. kidney and pancreas). 2. eGFR<30 mL/min/1.73 m². 3. Advanced Transplant Glomerulopathy (CG3). 4. Previous allergic reactions to monoclonal antibodies. 5. Lactating or pregnant females. 6. Women of child-bearing age who are not willing or able to practice FDA-approved forms of contraception during study and for 5 months after last dose. 7. HIV-positive subjects. 8. Subjects who test positive for HBV by HBVeAg/DNA or HCV infection [positive Anti-HCV (EIA) and confirmatory HCV MBA]. 9. Subjects with latent or active TB. Subjects must have negative Quantiferon TB gold test result. 10. Recent recipients of any licensed or investigational live attenuated vaccine(s) within two months of the screening visit j) A significantly abnormal general serum screening lab result defined as a WBC<3.0×10³/ml, a Hgb<8.0 g/dL, a platelet count<100×10³/ml, an SGOT or SGPT>3× upper limit normal. 11. Individuals deemed unable to comply with the protocol. 12. Subjects with active CMV or EBV infection as defined by CMV-specific serology (IgG or IgM) and confirmed by quantitative PCR with or without a compatible illness. 13. Use of investigational agents within 4 weeks of participation. 14. History or active Inflammatory Bowel Disease or Diverticular Disease or gastrointestinal perforation. 15. Recent infection (within past 6 weeks of screening) requiring any antibiotic use (oral, parenteral or topical). 16. Present or previous (within 5 years) malignancy except for basal cell carcinoma, fully excised squamous cell carcinoma of the skin or non-recurrent (within 5 years) cervical carcinoma-in-situ.

For purpose of this study, ABMR is defined as—
Deterioration of allograft function in a transplant recipient measured by eGFR (defined as an eGFR>30 cc/min reduction from baseline).
Association with the presence of DSA (usually increasing in strength) measured by luminex techniques.
Biopsy evidence of cABMR and TG by biopsy by Banff 2015 criteria.

Treatment of Allograft Rejection Episodes During the Study

Biopsy-proven rejection episodes that occur during the study are treated with “pulse” methylprednisolone (10 mg/kg/day, max 1000 mg for >100 kg for 3 days) and anti-thymocyte globulin (1.5 mg/kg daily×4) for cell-mediated rejection episodes that are unresponsive to pulse steroids. Patients experiencing recurrent ABMR episodes after study drug treatment, will initially receive pulse methylprednisolone (10 mg/kg/day, max 1000 mg for >100 kg) IV daily×3 doses then, depending on severity, IVIG 10% solution 2 gm/kg (max 140 g for >70 kg) IV×1 dose followed by rituximab (375 mg/m² rounded to the nearest 100 mg) IV×1 dose three to five days after last IVIG dose. In cases where rapid deterioration of allograft function is seen and/or thrombotic microangiopathy diagnosed, the patient will receive plasma exchange×3-5 sessions followed by anti-C5 (Eculizumab) IV weekly×4 weeks (1200 mg week #1 followed by 900 mg/weekly for 3 additional weeks). Efficacy of therapy will be assessed by determining renal function improvement, monitoring DSA responses and repeat allograft biopsies, if needed.

Monitoring AE/SAEs Post-Transplant in Highly-Sensitized Patients

Adverse events (AEs) and serious adverse events will be monitored post-ABMR treatment with clazakizumab. These include careful attention to infectious complications potentially associated with clazakizumab therapy. Infectious complications associated with IVIG+rituximab desensitization and alemtuzumab induction therapy followed by maintenance therapy with tacrolimus, MMF and prednisone have been assessed by the Applicant. Data showed that the use of a desensitization protocol followed with alemtuzumab induction does not increase the risk for common or serious infections post-transplant compared to a low risk group of patients. Serious infections were defined as any viral infection and fungal or bacterial infections requiring i.v. antibiotics or hospitalizations. Thus risk for infections in the instant Study (clazakizumab) after treatment will likely be similar and comparable to non-sensitized patients. All patients entered into this study are required to be vaccinated for data show that the use of this desensitization protocol followed with alemtuzumab induction does not increase the risk for common or serious infections post-transplant compared to a low risk group of patients. Serious infections were defined as any viral infection and fungal or bacterial infections requiring i.v. antibiotics or hospitalizations. Thus risk for infections in the study group (clazakizumab) after ABMR treatment will likely be similar and comparable to non-sensitized patients. All patients entered into this study are required to be vaccinated for Streptococcus pneumoniae.

Patient and Methods

Since February 2018, eight adult patients with biopsy proven cABMR+transplant glomerulopathy (TG) and DSA+ were enrolled in a phase I/II, single-center, open-label exploratory study. All patients received clazakizumab 25 mg subcutaneous injections monthly for 6 doses followed by a 6-month protocol biopsy. Patients were monitored for DSA relative intensity scores [(RIS); 0=No DSA; 2=<5000MFI(weak); 5=5000-10⁴MFI (moderate); 10=>10⁴MFI (strong)], renal function, C-reactive protein (CRP) levels, and T-regulatory cell (Treg) response.

All study patients, regardless of their cytomegalovirus (CMV) status, receive IV ganciclovir while inpatients and valganciclovir as outpatients for 6 months post kidney transplant, with dose adjustments for renal function. Fungal prophylaxis was accomplished with fluconazole 100 mg daily for 1 month post-transplant. Pneumocystis jirovecii pneumonia and bacterial prophylaxis is accomplished with trimethoprim 80 mg and sulfamethoxazole 400 mg daily for 12 months post-transplant. No additional prophylaxis will be needed for patients who are enrolled in this trial more than one year from transplant.

Clazakizumab vials should be stored at ≤−20° C. (−4° F.) with protection from light. The drug product will be administered undiluted at a concentration of 25 mg/mL. Prepared syringes may be stored for up to 24 hours in a refrigerator, 2°-8° C. (36°-46° F.), and up to 4 hours of the 24 hours may be at room temperature, 15°-25° C. (59°-77° F.). The prepared syringes should be protected from light. Prior to administration, clazakizumab should reach room temperature by storing unrefrigerated for 30 to 60 minutes before use.

Results

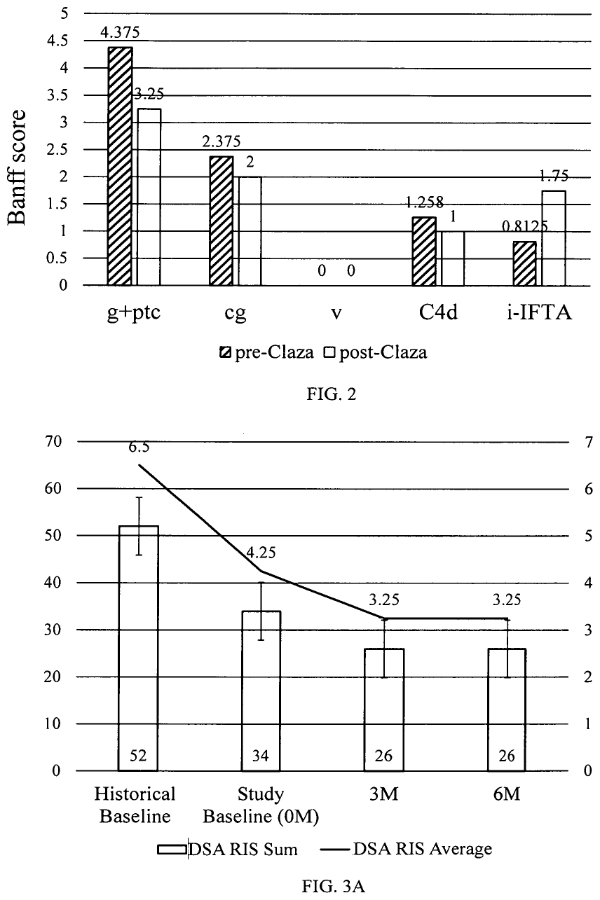

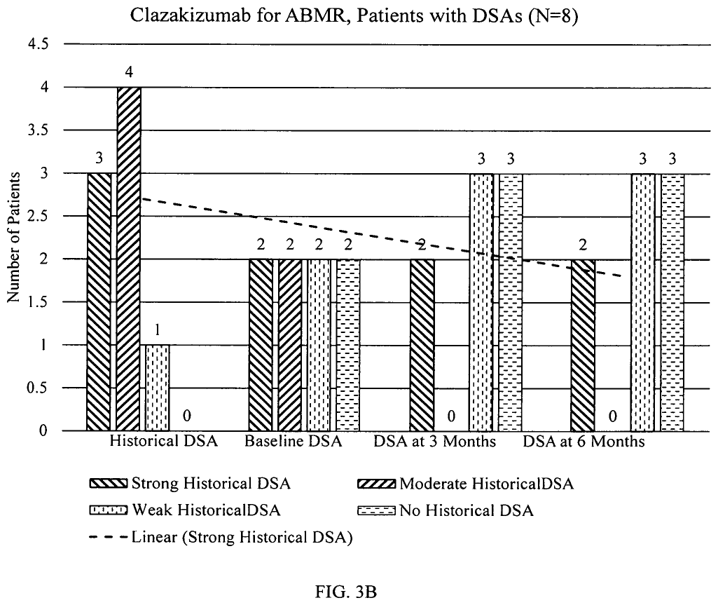

All patients showed marked reductions in CRP levels post-clazakizumab. 100% of patients' DSAs were class II (DQ, 75%). After 6 months, reductions in mean relative intensity score of DSA level (DSA-RIS) were observed (6.50±3.07 historical vs 3.25±4.27 at 6M, p=0.637), while mean GFRs remained stable (43.25±7.63 ml/min at 0M vs. 41.35±8.54 ml/min at 6M, p=0.647). 6-month biopsies exhibited the following changes in Banff scoring: glomerulitis+peritubular capillaritis (g+ptc) 4.38 at 0M to 3.38 at 6M (p=0.0097), glomerular double contours (cg) 2.13 at 0M to 1.88 at 6 M (p=0.718), intimal arteritis (v) 0 to 0 at M0 and 6M, complement-4d protein (C4d) 1.50 at 0M to 1.25 at 6M (p=0.693), i-IFTA (immunodominant interstitial fibrosis/tabular atrophy) 0.563 at 0M to 1.75 at 6M (p=0.036). (FIGS. 2 and 3A). Treg cells tended to increase at 3M. No serious adverse events have occurred to the preparation of this patent application. In calculation of DSA sum MFI (scale), a patient's MFI>10k was considered 10, MFI between 5k and 10k was considered 5, weak NM was considered 2, and no MFI was considered 0.

cABMR+TG patients treated with clazakizumab showed stabilization of renal function and improvements in DSA RIS. Biopsy findings showed trends in reduced g+ptc, cg and C4d scores.

Detailed monthly SCr eGFR data is shown in Table 1. Levels of IgG, CRP, and Treg at various time points in the study are shown in Table 2. Table 3 shows the detailed disease conditions, prior treatment history and notes from past biopsies of the eight patients in the study. Detailed DSA levels at various time points corresponding to FIG. 3B is provided in Table 4.

### Example 2. Clazakizumab Treatment of Patients with cABMR Reduces Total Immunoglobulin (Ig) and Anti-HLA IgG Antibody Levels

Clazakizumab is 3-120 times more potent than Tocilizumab in inhibiting IL-6/IL-6R signaling in vitro. In the study of improving cABMR using clazakizumab in sensitized kidney transplant patients, levels were measured of IgG, IgM, IgA, IgG subclasses, anti-HLA IgG and donor specific antibody (DSA) levels pre- and post-clazakizumab treatment.

Plasma samples obtained pre- & at 6 months post-clazakizumab (25 mg SQ, monthly) from 7 patients with cABMR were tested for total IgG, IgM, IgA and IgG₁₋₄ subclasses by ELISA. Anti-HLA IgG and DSAs were measured by single bead Luminex assay. The anti-HLA IgG and DSA (class I & class II) levels were expressed as a relative intensity score; Score 10, 5, 2 and 0 for MFI>10K, 5K-10K, <5K and no HLA antibody, respectively, are given to each detected antibody, and the sum of these are the final score for plasma with multiple HLA antibodies.

Total IgG, IgG₁ and IgG₂ significantly decreased post-clazakizumab, while no reduction was seen in total IgM, IgA, IgG3 and IgG4 (Table 5). Anti-HLA IgG was also significantly reduced post-clazakizumab; 4 of 7 patients (57%) showed reduction post-clazakizumab and the remaining 3 patients with low scores (<6) no change. DSA was reduced post-clazakizumab in 2 patients, and 3 with DSA and 2 without DSA showed no change.

As such, clazakizumab suppressed Ig production including total IgG, IgG₁, IgG2, anti-HLA IgG and DSA likely due to non-specific B cell suppression by blocking the effect of IL-6. This is believed to contribute to improvement of cABMR in this patient population.

### Example 3. Role of IL-6 in Mediation of ABMR

We investigated the role of IL-6 overexpression in the mediation of ABMR, and measured serum cytokine levels in peripheral blood of end-stage renal disease (ESRD) patients awaiting kidney transplant.

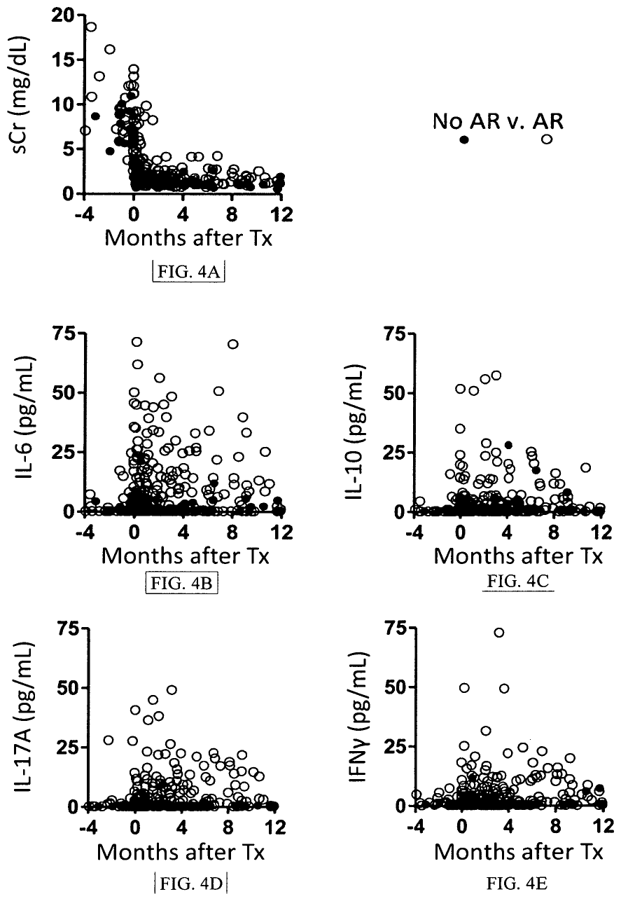

FIG. 4B shows the IL-6 levels are quite low in patients with quiescent allografts. FIG. 4G shows patients with ABMR show significant elevations of IL-6 serum levels in concert with ABMR onset. This data indicates that elevations of serum IL-6 levels could be used as an early marker for allograft dysfunction mediated by antibody injury.

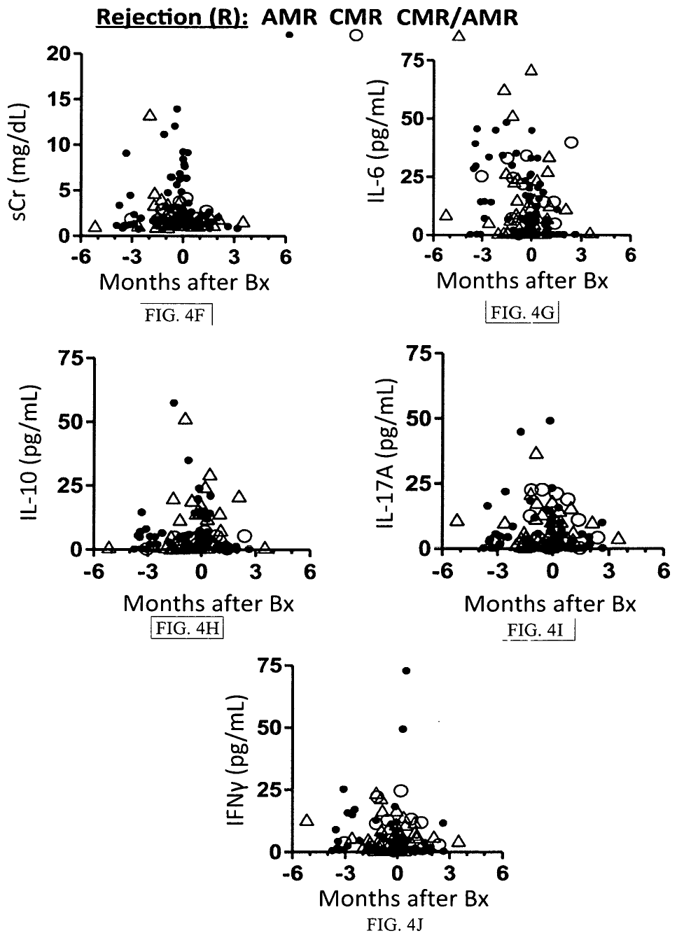

Next Applicant determined the expression of IL-6 in the biopsies of patients undergoing allograft rejection. Renal biopsy materials from patients with normal kidneys, patients with cellular rejection and patients with ABMR were examined. Sections were stained with anti-sera directed at IL-6 and evaluated by morphometric scanning microscopy. FIG. 5A shows that the number of IL-6+ cells were significantly increased in biopsies demonstrating ABMR. FIG. 5B quantifies from morphometric analysis the number of IL-6+ cells per mm² of tissue per staining in native kidneys (native, n=6 with thin basement membrane disease), transplants without rejection (tx, n=9), transplants with cell mediated rejection (CMR, n=12), and antibody-mediated rejection (ABMR, n=11). This indicates a possible role for IL-6/IL-6R interactions in mediating ABMR and enhanced DSA production. Taken together with the data on elevated levels of IL-6 in the sera of patients with ABMR, these findings are believed to show that IL-6 plays an important role in antibody-mediated injury to allografts and that IL-6 blockade is a potentially important therapy in management of ABMR and even cABMR and TG.

### Example 4. Clazakizumab-Treated cABMR+TG Patients Showed Stabilization of Renal Function and Improvement in DSA Relative Intensity Score after 18 Months of Therapy

In the study in Example 1, all patients received clazakizumab 25 mg subcutaneous injections monthly for 6 doses, underwent a 6-month protocol biopsy, followed by 6 monthly 25 mg doses. After 12 months of therapy, patients were able to enter a long-term extension (LTE) to receive clazakizumab 25 mg subcutaneously every other month. Patients were monitored for DSA relative intensity scores (RIS), renal function, CRP levels, and T-regulatory (Treg) cell responses. When no DSA is detected, RIS=0; when DSA mean fluorescence intensity is greater than 0 and ≤5000, (a weak MFI), RIS=2; when DSA mean fluorescence intensity is between 5000 and 10⁴, (a moderate WI), RIS=5; when DSA mean fluorescence intensity is ≥10⁴, (a strong MFI), RIS=10.

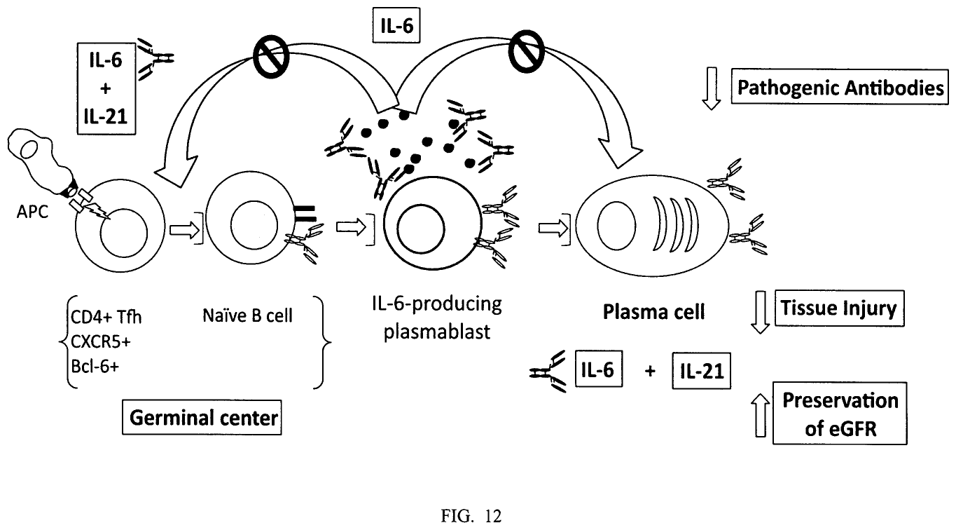

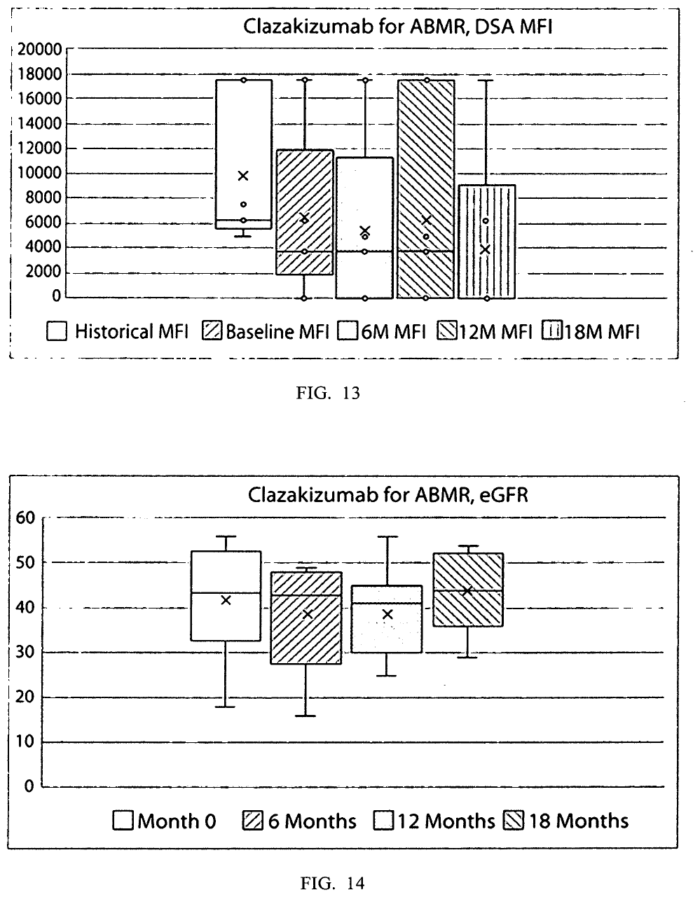

Eight patients continued to undergo clazakizumab therapy, seven of whom have transitioned to LTE dosing (N=5 have undergone 18 months of therapy). Two patients were withdrawn from the study to date, one patient progressed to graft failure and withdrew after four months of therapy, and one patient preferred to return to Tocilizumab (anti-IL6-R) therapy after 7 months. All patients showed marked reductions in CRP levels post-clazakizumab (1.11±1.25 at 0-month vs 0.43±0.17 at 12-month, p=0.31). After 12 months, reductions in mean DSA-RIS were sustained (6.40±3.31 historical vs 3.43±4.58 at 12-month, p=0.14) and in five patients, DSA-RIS remained reduced at 18 months (6.40±3.31 vs 2.50±4.18 at 18-month, p=0.06); see FIG. 13 for DSA WI. 100% of patients' DSAs were class II (DQ, 80%). Mean eGFRs remained stable at 12 months (41.90±12.09 mL/min at 0-month vs. 38.86±10.42 mL/min at 12-month, p=0.60); and 18-month in 5 patients (41.90±12.09 mL/min at 0-month vs 44±9.51 mL/min at 18-month, p=0.74); see FIG. 14. Treg cells tended to increase at 12 months (2.39±1.02% at 0M vs 3.30±1.13% at 12M, p=0.12). No serious adverse events were considered directly related to drug.

cABMR+TG patients treated with clazakizumab showed stabilization of renal function and improvements in DSA RIS after 18 months of therapy.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

TABLE 1
Monthly SCr eGFR levels of eight patients (all Caucasians,
01-07 male, 08 female) in the study.

| Patient | | | TX | Scr | eGFR | SCr | eGFR | SCr | eGFR | SCr |
|---|---|---|---|---|---|---|---|---|---|---|
| ID | Age | Tx | # | baseline | baseline | 1 M | 1 M | 2 M | 2 M | 3 M |
| CLAZA | 70 | LURT | 4 | 1.5 | 46 | 1.4 | 50 | 1.3 | 55 | 1.56 |
| BMR01 | | | | | | | | | | |
| CLAZA | 67 | DD | 4 | 1.76 | 39 | 1.5 | 47 | 1.71 | 40 | 1.65 |
| BMR02 | | | | | | | | | | |
| CLAZA | 40 | LRRT | 1 | 2.11 | 35 | 2 | 37 | 2.19 | 33 | 2.27 |
| BMR03 | | | | | | | | | | |
| CLAZA | 57 | LURT | 1 | 1.4 | 52 | 1.4 | 52 | 1.47 | 49 | 1.65 |
| BMR04 | | | | | | | | | | |
| CLAZA | 62 | LURT | 1 | 1.5 | 47 | 1.3 | 56 | 1.44 | 50 | 1.63 |
| BMR05 | | | | | | | | | | |
| CLAZA | 21 | DD | 1 | 2 | 42 | 2.1 | 40 | 2.55 | 32 | 2.39 |
| BMR06 | | | | | | | | | | |
| CLAZA | 51 | LRRT | 1 | 1.4 | 53 | 1.4 | 53 | 1.57 | 47 | 1.6 |
| BMR07 | | | | | | | | | | |
| CLAZA | 50 | DD | 1 | 1.7 | 32 | 1.8 | 30 | 2.01 | 26 | 1.82 |
| BMR08 | | | | | | | | | | |
| | | | | Avg | Avg | | | | | |
| | | | | SCr | eGFR | | | | | |
| | | | | 1.67 | 43.25 | | | | | |
| | | | | 0.27 | 7.63 | | | | | |
| | | | | t | 0.65 | | | | | |
| | | | | test | | | | | | |

| Patient | eGFR | SCr | eGFR | SCr | eGFR | SCr | eGFR | Avg | Avg |
|---|---|---|---|---|---|---|---|---|---|
| ID | 3 M | 4 M | 4 M | 5 M | 5 M | 6 M | 6 M | SCr | eGFR |
| CLAZA | 44 | 1.43 | 46 | 1.53 | 45 | 1.4 | 50 | 1.4 | 48 |
| BMR01 | | | | | | | | | |
| CLAZA | 42 | 1.64 | 42 | 1.6 | 43 | 1.52 | 46 | 1.6 | 43 |
| BMR02 | | | | | | | | | |
| CLAZA | 32 | 2.19 | 33 | 2.22 | 33 | 2.2 | 33 | 2.2 | 34 |
| BMR03 | | | | | | | | | |
| CLAZA | 43 | 1.59 | 45 | 1.6 | 45 | 1.73 | 41 | 1.6 | 46 |
| BMR04 | | | | | | | | | |
| CLAZA | 43 | 1.38 | 52 | 1.45 | 49 | 1.36 | 53 | 1.4 | 51 |
| BMR05 | | | | | | | | | |
| CLAZA | 35 | 2.57 | 32 | 2.48 | 33 | 2.3 | 48 | 2.4 | 37 |
| BMR06 | | | | | | | | | |
| CLAZA | 46 | 1.5 | 49 | 1.65 | 44 | 1.71 | 42 | 1.6 | 47 |
| BMR07 | | | | | | | | | |
| CLAZA | 29 | 2.02 | 26 | 2.45 | 21 | 2.29 | 23 | 2.1 | 26 |
| BMR08 | | | | | | | | | |
| | | | | | | | | Avg | Avg |
| | | | | | | | 6 M | SCr | eGFR |
| | | | | | | | | 1.78 | 41.35 |
| | | | | | | | Standard | 0.37 | 8.54 |
| | | | | | | | Deviation | | |

TABLE 2
Levels of IgG, CRP, and Treg at various time points of the eight patients in the study.

| | | | | IgG | | | CRP | | | | | | Treg | | |
|---|---|---|---|---|---|---|---|---|---|---|---|---|---|---|---|
| | | | Tx | base- | IgG | IgG | base- | CRP | CRP | CRP | CRP | CRP | Base- | Treg | Treg |
| Study ID | Trx | Date of tx | # | line | 3 M | 6 M | line | 1 M | 2 M | 3 M | 4 M | 5 M | line | 3 M | 6 M |
| CLAZABMR01 | LURT | Jun. 14, 2013 | 4 | 1030 | 1029 | 991 | 1.1 | ND | 0.7 | 0.5 | 0.3 | 0.5 | 1.3 | 1.3 | 1.1 |
| CLAZABMR02 | DD | Jan. 29, 2015 | 4 | 669 | 603 | 592 | 1.3 | ND | 0.2 | 0.4 | 0.4 | 0.2 | 2.2 | 1.7 | 1.8 |
| CLAZABMR03 | LRRT | Dec. 21, 1995 | 1 | 796 | nd | 741 | 0.3 | 0.4 | 0.4 | 0.4 | 0.3 | 0.2 | 2.2 | 3.1 | 1.9 |
| CLAZABMR04 | LURT | Aug. 27, 2010 | 1 | 725 | nd | 538 | 0.3 | 0.5 | 0.3 | 0.7 | 0.3 | 0.2 | 3.9 | 4.5 | 1.8 |
| CLAZABMR05 | LURT | Jan. 20, 2015 | 1 | 778 | 721 | 679 | 4.2 | 0.5 | 0.5 | 0.4 | 0.5 | 0.3 | 1.7 | 2.2 | 1.9 |
| CLAZABMR06 | DD | Jul. 10, 2004 | 1 | 755 | 935 | 332 | 0.7 | 0.5 | 0.6 | 0.2 | 0.2 | 0.4 | 4.1 | 4.4 | 2.1 |
| CLAZABMR07 | LRRT | Jun. 24, 2008 | 1 | 1700 | 983 | 860 | 1.5 | 0.5 | 0.2 | 0.3 | 0.3 | 0.3 | 1.8 | 1.9 | 2.1 |
| CLAZABMR08 | DD | Jul. 8, 2014 | 1 | 767 | 610 | pending | 0.3 | 0.2 | 0.2 | 0.2 | 0.2 | pending | 2.3 | 1.9 | pending |
| | | | Avg | 902.5 | 814 | 676 | 1.2 | 0.4 | 0.4 | 0.4 | 0.3 | 0.3 | 2.4 | 2.6 | 1.8 |
| | | | | | | Ttest | 0.07 | | | | | | | | |
| | | | | | | CRP | | | | | | | | | |

TABLE 3
Brief medical history of the eight patients in the study.

| Patient | | | Tx | Tx | Historical | | | | | |
|---|---|---|---|---|---|---|---|---|---|---|
| ID | Dx | Tx | Date | # | DSA | MFI | Rating | Treatment History | Past Biopsies | C4D+ |
| CLAZA | chronic | LURT | Jun. 14, | 4 | dnDSA | 5000- | 5 | des at tx: plex + | June 2013) mild acute | |
| BMR01 | glomerulo- | | 2013 | | DQ1*05 | 6250 | | IVIG + ritux | tubular injury, no | |
| | nephritis | | | | | | | September 2017) | evidence of rejection | |
| | and medullary | | | | | | | ivig + ritux | Jun. 6, 2017, which | |
| | sponge kidney | | | | | | | | demonstrated chronic, | |
| | | | | | | | | | active AMR with | |
| | | | | | | | | | glomerulitis, and TG. | |
| | | | | | | | | | Only mild IFTA | |
| CLAZA | hypertensive | DD | Jan. 29, | 4 | DP17 | weak | 2 | actemra x6 at trx, | May 16, 2016 that | |
| BMR02 | nephrosclerosis | | 2015 | | | | | then 6 M post trx | revealed chronic, | |
| | | | | | | | | June 2016 | active AMR, | |
| | | | | | | | | gazyva x 1 November | borderline | |
| | | | | | | | | 2017) 1 g obi x 1 | inflammatory | |
| | | | | | | | | | infiltrate, and | |
| | | | | | | | | | FSGS (likely secondary). | |
| CLAZA | congenital | LRRT | Dec. 21, | 1 | dnDSA | >17500 | 10 | September 2011: | BX- CMR September 2006. | |
| BMR03 | hypoplastic | | 1995 | | DQ7 | | | IVIG and rituximab. | Bx September 2011, TG, | |
| | kidneys | | | | | | | November 2017: | CNI toxicity | |
| | | | | | | | | PLEX x 3, followed | Apr. 3, 2015 TG | |
| | | | | | | | | by IVIG split dose, | chronic ABMR; focal | |
| | | | | | | | | and Rituxan. | glomerulitis, secondary | |
| | | | | | | | | | FSGS, severe arteriolar | |
| | | | | | | | | | hyalinization consistent | |
| | | | | | | | | | with CNI toxicity | |
| CLAZA | medullary cystic | LURT | Aug. 27, | 1 | DR51 | 5000- | 5 | February 2016: | Feb. 26, 2016 that | yes |
| BMR04 | kidney disease | | 2010 | | (dnDSA) | 6250 | | ivig + ritux | revealed acute/active | |
| | | | | | | | | October 2017: | AMR and mild arterio- | |
| | | | | | | | | ivig | and arteriolosclerosis | |
| | | | | | | | | | October 2017: features | |
| | | | | | | | | | of waek C4d positivity | |
| | | | | | | | | | and focal peritubular | |
| | | | | | | | | | capillaritis and focal | |
| | | | | | | | | | glomerulitis | |
| CLAZA | htn | LURT | Jan. 20, | 1 | dnDSA | >17500 | 10 | March 2016) | Mar. 16, 2016 revealed | Yes |
| BMR05 | | | 2015 | | DQ8 | | | ivig + ritux | Banff 1A CMR and | |
| | | | | | | | | November 2016) | chronic active AMR; | |
| | | | | | | | | plex + ritux | DIFF USE, MILD | |
| | | | | | | | | February 2017) | PERITUBULAR | |
| | | | | | | | | ritux | CAPILLARY | |
| | | | | | | | | | C4d STAINING; | |
| | | | | | | | | | SECONDARY FSGS | |
| CLAZA | posterior | DD | Jul. 10, | 1 | dnDSA | strong | 10 | 2011) RITUX + | 2011) Bx showed evidence | yes |
| BMR06 | urethral valves | | 2004 | | DQ7 | | | IVIG | of CMR + ABMR February | |
| | with reflux, | | | | | | | July 2015) | 2013 demonstrated AMR | |
| | obstructive | | | | | | | IVIG + ACTEMRA | July 2015) ACUTE cABMR | |
| | uropathy, and | | | | | | | June 2016 | May 2016) chronic active | |
| | pyelonephritis | | | | | | | GAZYVA X1G | ABMR, FSGS, mild if/ta | |
| | | | | | | | | | January 2018) chronic | |
| | | | | | | | | | active ABMR, secondary | |
| | | | | | | | | | FSGS, mod if/ta | |
| CLAZA | granulomatosis | LRRT | Jun. 24, | 1 | dnDSA | mod- | 5 | pretransplant | Nov. 1, 2016 that | Yes |
| BMR07 | with poly | | 2008 | | DQ2 | erate | | treatment with | revealed mildly to | |
| | angiitis | | | | | | | IVIG and | moderately active and | |
| | | | | | | | | rituximab to | chronic AMR, minimal if/ta | |
| | | | | | | | | minimize | Mar. 20, 2018 demonstrated | |
| | | | | | | | | recurrence of | chronic active AMR, | |
| | | | | | | | | his ANCA- | C4d positive | |
| | | | | | | | | positive | | |
| | | | | | | | | glomerulonephritis | | |
| | | | | | | | | November 2016 | | |
| | | | | | | | | plex 4, ritux + | | |
| | | | | | | | | ivig | | |
| | | | | | | | | May 2017: PLEX x | | |
| | | | | | | | | 5, followed by | | |
| | | | | | | | | IVIG + actemra | | |
| | | | | | | | | BEFORE STUDY- | | |
| | | | | | | | | PLEX + IVIG | | |
| CLAZA | unknown | DD | Jul. 08, | 1 | dnDSA | mod- | 5 | December 2014) | Dec. 9, 2014 Banff 1B | |
| BMR08 | etiology | | 2014 | | DQ4 | erate | | IVIG + ritux | acute CMRb glomerular | |
| | | | | | | | | August 2016) | and tubular catheter and | |
| | | | | | | | | ivig + gazyva | microvascular inflammation | |
| | | | | | | | | July 2017) | consistent with ABMR; | |
| | | | | | | | | ivig + actemra x 6 | Jul. 18, 2017 was | |
| | | | | | | | | | consistent with | |
| | | | | | | | | | active cABMR. | |
| | | | | | | | | | moderate/severe IFTA | |

TABLE 4
Levels of DSA at various time points of the eight patients in the study. (Patient CLAZABMR03 had strong AT1R, >40IU).

| | Baseline | | | | | | | | | | | |
|---|---|---|---|---|---|---|---|---|---|---|---|---|
| Patient | (Study | | | | DSA | | | | DSA | | | |
| ID | entry) | Date | MFI | Rating | 3 M | Date | MFI | Rating | 6 M | Date | MFI | Rating |
| CLAZA | DQ1*O5 | Feb. 21, | 5000- | 5 | DQ1*O5 | May 23, | 2500- | 2 | DQ1*05 | Sep. 12, | 3750- | 2 |
| BMR01 | | 2018 | 6250 | | | 2018 | 3750 | | | 2018 | 5000 | |
| CLAZA | no DSA | Feb. 21, | 0 | 0 | no DSA | May 23, | 0 | 0 | no DSA | Sep. 26, | 0 | 0 |
| BMR02 | | 2018 | | | | 2018 | | | | 2018 | | |
| CLAZA | DQ7 | Feb. 21, | >17500 | 10 | DQ7 | May 2, | >17500 | 10 | DQ7 | Sep. 27, | >17500 | 10 |
| BMR03 | | 2018 | | | | 2018 | | | | 2018 | | |
| CLAZA | no DSA | Feb. 21, | 0 | 0 | no DSA | May 3, | 0 | 0 | no DSA | Sep. 27, | 0 | 0 |
| BMR04 | | 2018 | | | | 2018 | | | | 2018 | | |
| CLAZA | DQ8 | Mar. 19, | 2500- | 2 | DQ8 | Jun. 27, | 2500- | 2 | DQ8 | Jun. 27, | 2500- | 2 |
| BMR05 | | 2018 | 3750 | | | 2018 | 3750 | | | 2018 | 3750 | |
| CLAZA | DQ7 | May 2, | >17500 | 10 | DQ7 | Jul. 6, | >17500 | 10 | DQ7 | Aug. 31, | >17500 | 10 |
| BMR06 | | 2018 | | | | 2018 | | | | 2018 | | |
| CLAZA | DQ2 | Feb. 13, | 5000- | 5 | DQ2 | Jun. 21, | 3750- | 2 | DQ2 | Sep. 13, | 3750- | 2 |
| BMR07 | | 2018 | 6250 | | | 2018 | 5000 | | | 2018 | 5000 | |
| CLAZA | DQ4 | May 30, | 2500- | 2 | DQ4 | Aug. 2, | 0 | 0 | DQ4 | Oct. 25, | 0 | 0 |
| BMR08 | | 2018 | 3750 | | | 2018 | | | | 2018 | | |

TABLE 5
Levels of total IgG, IgM, IgA and
IgG₁₋₄ subclasses quantified by ELISA.

| | Pre-clazakizumab | Post-clazakizumab |
|---|---|---|
| Total IgG (mg/ml) | 15.3 ± 3.4 | 13.0 ± 2.6* |
| IgG1 (mg/ml) | 11.4 ± 6.2 | 10.9 ± 4.0^{#} |
| IgG2 (mg/ml) | 2.7 ± 1.5 | 1.7 ± 1.2* |
| IgG3 (mg/ml) | 0.7 ± 0.6 | 0.6 ± 0.6 |
| IgG4 (mg/ml) | 0.1 ± 0.1 | 0.1 ± 0.1 |
| Total IgM (mg/ml) | 3.2 ± 1.2 | 3.8 ± 1.1 |
| Total IgA (mg/ml) | 4.2 ± 0.9 | 4.2 ± 0.8 |
| Anti-HLA IgG (score) | 47 ± 47 | 43 ± 44* |
| DSA (score) | 3.4 ± 3.3 | 2.7 ± 3.4 |

*p < 0.0.5,
0.05 < ^{#}P < 0.1 vs. pre-clazakizumab

What is claimed is:

1. A method for treating or reducing the severity of antibody-mediated rejection (ABMR) of an organ transplant in a subject, comprising:
administering to the subject an effective amount of clazakizumab; an IL-6 binding fragment of clazakizumab; a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; or a conservatively substituted, added or deleted variant thereof.

2. The method of claim 1, wherein the subject is human leukocyte antigen (HLA)-sensitized before the administration.

3. The method of claim 1, wherein the subject is diagnosed with or exhibits symptoms of ABMR.

4. The method of claim 1, wherein the subject is diagnosed with or exhibits symptoms of chronic active ABMR, transplant glomerulopathy (TG), and is donor-specific antibodies (DSAs) positive.

5. The method of claim 1, wherein after the administration the subject has a reduced level of C-reactive protein, reduced DSA level, reduced Banff scores in one or more of glomerulitis+peritubular capillaritis (g+ptc), glomerular double contours (cg) and complement-4d protein (C4d), or a combination thereof, compared to that before the administration.

6. The method of claim 1, wherein the subject has received a standard-of-care treatment which comprises intravenous immunoglobulin (IVIG) administration, rituximab administration, plasmapheresis, methylprednisolone administration, or a combination thereof, or has received an immunosuppressive agent comprising eculizumab, before the clazakizumab administration.

7. The method of claim 6, wherein the subject's response to the standard-of-care treatment or to the immunosuppressive agent is ineffective.

8. The method of claim 1, wherein the organ is a kidney.

9. The method of claim 1, wherein the organ is one or more of heart, liver, lung, pancreas, and intestine.

10. The method of claim 1, wherein clazakizumab, the IL-6 binding fragment of clazakizumab, or the polypeptide is administered subcutaneously or intravenously.

11. The method of claim 1, wherein clazakizumab, the IL-6 binding fragment of clazakizumab, or the polypeptide is administered subcutaneously at an average dose of about 0.1-1 mg/month, 1-5 mg/month, 5-10 mg/month, 10-20 mg/month, 20-30 mg/month, or 30-40 mg/month for a minimum of 6 months.

12. The method of claim 1, wherein clazakizumab, the IL-6 binding fragment of clazakizumab, or the polypeptide is administered at about monthly intervals for 1 month, 2 months, 3 months, 4 months, 5 months, 6 months, 7 months, 8 months, 9 months, 10 months, 11 months, 12 months, 13 months, 14 months, 15 months, 16 months, 17 months, 18 months or longer.

13. The method of claim 1, further comprising administering one or more anti-infectious agents to the subject.

14. The method of claim 13, wherein the anti-infectious agent comprises ganciclovir, valganciclovir, fluconazole, trimethoprim, sulfamethoxazole, or a combination thereof.

15. A method for reducing C-reactive protein and/or donor-specific antibody in a human subject having antibody-mediated rejection of an allograft transplant, comprising:
administering to the subject a pharmaceutical composition comprising an effective amount of clazakizumab; an IL-6 binding fragment of clazakizumab; or a polypeptide having V_{H} polypeptide containing CDR1, CDR2, or CDR3, or a combination thereof, which respectively are contained in SEQ ID NO: 1 for CDR1 of V_{H}, SEQ ID NO: 2 or SEQ ID NO:3 for CDR 2 of V_{H}, SEQ ID NO: 4 for CDR3 of V_{H}, and having V_{L} polypeptide containing CDR1, CDR2, and CDR3 polypeptides which respectively are contained in SEQ ID NO: 5, 6, and 7; and one or more pharmaceutically acceptable excipients.

16. The method of claim 15, further comprising administering a standard-of-care treatment which comprises intravenous immunoglobulin (IVIG) administration, rituximab administration, plasmapheresis, or a combination thereof.

17. The method of claim 15, further comprising administering an anti-infectious agent.

18. The method of claim 1, further comprising selecting a subject diagnosed with chronic active ABMR, transplant glomerulopathy (TG), and who is donor-specific antibodies (DSAs) positive, before the administration.

19. The method of claim 1, further comprising conducting with the subject one or more times of immune monitoring comprising assaying a blood sample of the subject to quantify levels of C-reactive protein, regulatory T cells, Tfh, Th17, B-cell, IL-6, plasma cells, plasmablast IgG, or a combination thereof.

20. The method of claim 1, further comprising measuring the amount of glomerular filtration rate, DSA or both, after the administration.


SEQUENCE LISTING (12 sequences)

SEQ ID NO: 1 - protein, 5 aa, Artificial Sequence
  VH CDR1
NYYVT

SEQ ID NO: 2 - protein, 16 aa, Artificial Sequence
  VH CDR2
IIYGSDETAYATWAIG

SEQ ID NO: 3 - protein, 16 aa, Artificial Sequence
  Another VH CDR2
IIYGSDETAYATSAIG

SEQ ID NO: 4 - protein, 12 aa, Artificial Sequence
  VH CDR3
DDSSDWDAKFNL

SEQ ID NO: 5 - protein, 11 aa, Artificial Sequence
  VL CDR1
QASQSINNELS

SEQ ID NO: 6 - protein, 7 aa, Artificial Sequence
  VL CDR2
RASTLAS

SEQ ID NO: 7 - protein, 12 aa, Artificial Sequence
  VL CDR3
QQGYSLRNIDNA

SEQ ID NO: 8 - protein, 166 aa, Artificial Sequence
  VH
METGLRWLLLVAVLKGVQCQSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 9 - protein, 109 aa, Artificial Sequence
  Another VH
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNL

SEQ ID NO: 10 - protein, 109 aa, Artificial Sequence
  Another VH
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNL

SEQ ID NO: 11 - protein, 163 aa, Artificial Sequence
  VL
MDTRAPTQLLGLLLLWLPGARCAYDMTQTPASVSAAVGGTVTIKCQASQSINNELSWYQQKPGQRPKLLIYRASTLASGVSSRFKGSGSGTEFTLTISDLECADAATYYCQQGYSLRNIDNAFGGGTEVVVKRTVAAPSVFIFPPSDEQLKSGTASVVCLLNN

SEQ ID NO: 12 - protein, 99 aa, Artificial Sequence
  Another VL
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNA